(19) United States
(12) Patent Application Publication
(10) Pub. No.: US 2006/0122179 A1
(43) Pub. Date: Jun. 8, 2006

(54) Methods and compositions using JNK inhibitors for treatment and management of central nervous system injury

(76) Inventors: Jerome B. Zeldis, Princeton, NJ (US); Herbert Faleck, West Orange, NJ (US); Donald C. Manning, Bloomsbury, NJ (US)

Correspondence Address: JONES DAY, 222 EAST 41ST ST, NEW YORK, NY, 10017, US

(21) Appl. No.: 11/286,128

(22) Filed: Nov. 22, 2005

Related U.S. Application Data
(60) Provisional application No. 60/630,598, filed on Nov. 23, 2004.

(51) Int. Cl. A61K 31/5377; A61K 31/4245; A61K 31/454; A61K 31/4196; A61K 31/416

(52) U.S. Cl. 514/232.5; 514/322; 514/383; 514/381; 514/364; 514/406

(57) ABSTRACT

Methods of treating, preventing and/or managing a central nervous system injury/damage and related syndromes are disclosed. Specific methods encompass the administration of a JNK inhibitor alone or in combination with a second active agent. Pharmaceutical compositions, single unit dosage forms, and kits suitable for use in methods of the invention are also disclosed.

This application claims the benefit of U.S. provisional application No. 60/630,598, filed Nov. 23, 2004, the contents of which are incorporated by reference herein in their entirety.

### 1. FIELD OF THE INVENTION

This invention relates to methods of treating, preventing and/or managing central nervous system injury/damage and related syndromes which comprise the administration of a JNK inhibitor, or a pharmaceutically acceptable salt, solvate, hydrate, stereoisomer, clathrate, or prodrug thereof.

### 2. BACKGROUND OF THE INVENTION

2.1. Central Nervous System Injury

Central nervous system (CNS) injury/damage can be classified into three categories: (a) CNS injury/damage caused by mechanical damage to the brain; (b) CNS injury/damage caused by reduced blood supply to the brain, which can occur in ischemic or hemorrhagic stroke, or as a result of hypoxia; and (c) CNS injury/damage related to the spinal cord injury caused by trauma, infection or toxicity.

Traumatic brain injury (TBI) is an example of mechanical damage, and one of the leading causes of death and lifelong disability in the United States today. Greenwald et al., Arch Phys. Med. Rehabil. 2003; 84 (3 Supp.1): S3. The pathophysiology of TBI can be separated into primary injury and secondary injury. Id., p. S4. Primary injury occurs at the time of impact, while secondary injury occurs after the impact secondary to the body's response to primary injury. Id. Each of primary and secondary injuries can be subdivided into focal and diffuse types. Id. Focal injury tends to be caused by contact forces, whereas diffuse injury is likely to be caused by noncontact, acceleration-deceleration, or rotational forces. Id.

Specific types of primary injury include scalp injury, skull fracture, basilar skull fracture, concussion, contusion, intracranial hemorrhage, subarachnoid hemorrhage, epidural hematoma, subdural hematoma, intraventricular hemorrhage, subarachnoid hemorrhage, penetrating injuries, and diffuse axonal injury. Primary focal injury is caused by cortical contusions and intracranial hematomas. Greenwald et al., p. S4. Contusions usually occur after direct injuries over bony prominences of the skull. The commonly affected areas are the orbitofrontal and anterotemporal regions. Id. Intracranial hematomas are divided into epidural hematomas, subdural hematomas, and subarachnoid hemorrhages. Id. Epidural hematomas result from rupture of the middle meningeal artery. Id. They cause focal injury by increasing pressure over a cortical region of the brain. Id. Subdural hematomas and subarachnoid hemorrhage occur as a result of disruption of the bridging vessels in their respective spaces. Id. Both cause focal injury due to increased intracranial pressure (ICP). Id.

Diffuse axonal injury (DAI) is caused by forces associated with acceleration-deceleration and rotational injuries. Greenwald et al., p. S5. This type of injury most commonly occurs during the high-impact collisions of motor vehicle accidents. The injury can also be due to contact sports. Id. DAI is an axonal shearing injury of the axons that is most often observed in the midline structures, including the parasagittal white matter of the cerebral cortex, the corpus callosum, and the pontine-mesencephalic junction adjacent to the superior cerebral peduncles. Id.

Posttraumatic syndrome may develop following traumatic injury. The syndromes include hydrocephalus, altered level of consciousness, headache, migraine, nausea, emesis, memory loss, dizziness, diplopia, blurred vision, emotional lability, sleep disturbances, irritability, inability to concentrate, nervousness, behavioral impairment, cognitive deficit, and epilepsy. Seizures are commonly observed with contusions, depressed skull fracture and severe head injury. Intracranial infections are another potential complication of TBI. When basilar skull fractures or cerebrospinal fluid fistulae is present, the risk of infection is increased. In addition, if a patient has a ventriculostomy for ICP monitoring, the risk of infection is also increased for either a ventriculitis or meningitis. The incidence of infection increases in penetrating cerebral injuries and open depressed skull fractures.

Other causes of CNS injury/damage include neurochemical and cellular changes, hypotension, hypoxia, ischemia, electrolyte imbalances, increased ICP with decreased cerebral perfusion pressure (CPP) and a risk of herniation. Greenwald et al., p. S6. Acute loss of circulation to an area of the brain results in ischemia and a corresponding loss of neurologic function. Classified as either hemorrhagic or ischemic, strokes typically manifest with the sudden onset of focal neurologic deficits, such as weakness, sensory deficit, or difficulties with language. Ischemic strokes have a heterogeneous group of causes, including thrombosis, embolism, and hypoperfusion, whereas hemorrhagic strokes can be either intraparenchymal or subarachnoid. As blood flow decreases, neurons cease functioning, and irreversible neuronal ischemia and injury begin at blood flow rates of less than 18 mL/100 mg/min.

The processes involved in stroke injury at the cellular level are referred to as the ischemic cascade. Within seconds to minutes of the loss of glucose and oxygen delivery to neurons, the cellular ischemic cascade begins. The process begins with cessation of the electrophysiologic function of the cells. The resultant neuronal and glial injury produces edema in the ensuing hours to days after stroke, causing further injury to the surrounding neuronal tissues.

Without being limited by theory, CNS injury or spinal cord injury can lead to activated glial cells (microglia or astrocytes) with subsequent release of cytokines, chemokines, and other mediators of inflammation, in addition to glutamate.

Spinal cord injury (SCI) is an insult to the spinal cord resulting in a change, either temporary or permanent, in its normal motor, sensory, or autonomic function. The annual incidence of SCI in various countries ranges from 15-40 cases per million population. C. H. Tator, Brain Pathology 5:407-413 (1995). Both clinical and experimental studies evidence that the spinal cord suffers from primary and secondary damage after acute SCI. Id., 407. Primary SCI arises from mechanical disruption, transection, extradural pathology, or distraction of neural elements. Id. This injury usually occurs with fracture and/or dislocation of the spine. However, primary SCI may occur in the absence of spinal fracture or dislocation. Penetrating injuries due to bullets or weapons may also cause primary SCI. Bumey et al., Arch Surg 128(5): 596-9 (1993). More commonly, displaced bone fragments cause penetrating spinal cord or segmental spinal nerve injuries. Extradural pathology may also cause primary SCI. Spinal epidural hematomas or abscesses cause acute cord compression and injury. Spinal cord compression from metastatic disease is a common oncologic emergency. Longitudinal distraction with or without flexion and/or extension of the vertebral column may result in primary SCI without spinal fracture or dislocation.

The pathophysiology of secondary SCI involves a multitude of cellular and molecular events which progress over the first few days after injury. C. H. Tator, Brain Pathology 5:407-413 (1995). The most important cause of secondary SCI is vascular injury to the spinal cord caused by arterial disruption, arterial thrombosis, and hypoperfusion due to shock. SCI can be sustained through ischemia from damage or impingement on the spinal arteries. SCI due to ischemia can occur during surgery where aortic blood flow is temporarily stopped.

Spinal cord injury can be caused by infections. Infections involving the spinal canal include epidural abscesses (infection in the epidural space), meningitis (infection of the meninges), subdural abscesses (infections of the subdural space), and intramedullary abscesses (infections within the spinal cord). Mechanisms of the infections include hematogenous spread from an extraspinal focus of infection, contiguous spread from an adjacent focus of infection, direct inoculation (i.e., penetrating trauma or postneurosurgery), and cryptogenic mechanisms (i.e., no documented extraspinal focus of infection). Bacteria, such as staphylococci and streptococci, are the most common organisms responsible for these infections. However, infections also may be viral, fungal, or caused by cysticercosis, Mycobacterium tuberculosis, Listeria monocytogenes, Toxoplasma gondii, or other parasites. Initially, the area of the bacterial nidus is infiltrated with polymorphonuclear cells, leading to a suppurative myelitis. This evolves into central necrosis and liquefaction, which can spread along the long spinal tracts. At the periphery of this infectious process, fibroblasts proliferate, and the central purulent area becomes encapsulated by fibrous granulation tissue. The most commonly affected area is the dorsal thoracic spinal cord.

Spinal cord injury can also be caused by toxicity. Tator, p. 408-9. One of the most compelling toxicity in spinal cord injury is the accumulation and subsequent damage exerted by the excitatory amino acid neurotransmitter. Glutamate induced excitotoxicity causes an elevation of intracellular calcium. Id. Raised intracellular calcium can in turn cause activation of calcium dependent proteases or lipases which cause further damage due to breakdown of cytoskeletal components including neurofilaments and dissolution of cell membranes. Id. The excess production of arachidonic acid and eicosanoids such as prostaglandins may be related to lipid peroxidation and oxygen free radicals. Id. The release of vasoactive eicosanoids from damaged neuronal membranes may in turn cause progressive posttraumatic ischemia by inducing vasospasm. Id. Endogenous opioids may also be involved in the secondary injury process either by their effects on the local or systemic circulation or by direct effects on the injured cord. Id.

Increased intracellular calcium appears to trigger neurotoxicity in a variety of ways. There are major electrolyte shifts between the extracellular and intracellular compartments and vice versa after spinal cord injury. Tator, p. 409. An excess of free intracellular calcium ions plays a fundamental role in mediating the pathogenesis of all neural injuries, but especially ischemia and traumatic injuries. Id., p. 410. After trauma, calcium can shift into neurons in a variety of ways such as through disrupted cell membranes, or by depolarization and entry through voltage sensitive calcium channels, or through receptor mediated calcium channels activated by glutamate. Id. Secondary ischemia can also increase intracellular calcium through glutamate release. Id.

Significant and progressive edema can follow spinal cord injury. Tator, p. 410. It is not known whether the edema is injurious in itself or whether it is an epiphenomenon of another injury mechanism such as ischemia or glutamate toxicity. Id. Edema can spread in the cord from the site of injury for a considerable distance rostrally and caudally in both experimental models and clinical cases. Id.

SCI are classified as complete or incomplete, based on the extent of injury, according to the American Spinal Injury Association (ASIA) Impairment Scale. In complete SCI, there is no sensory and motor function preserved in the lowest sacral segments. Waters et al., Paraplegia 29(9): 573-81(1991). In incomplete SCI, sensory or motor function is preserved below the level of injury including the lowest sacral segments. Waters et al., Archives of Physical Medicine and Rehabilitation 75(3): 306-11(1994). Incomplete cord lesions may evolve into more complete lesions. More commonly, the injury level rises one or two spinal levels during the hours to days after the initial event. Id.

Other classifications of SCI include central cord syndrome, Brown-Sequard syndrome, anterior cord syndrome, conus medullaris syndrome and cauda equina syndrome. Central cord syndrome is often associated with a cervical region injury leading to greater weakness in the upper limbs than in the lower limbs with sacral sensory sparing. Brown-Sequard syndrome involves a hemisection lesion of the cord, causing a relatively greater ipsilateral proprioceptive and motor loss with contralateral loss of sensitivity to pain and temperature. Anterior cord syndrome is often associated with a lesion causing variable loss of motor function and sensitivity to pain and temperature, while proprioception is preserved. Conus medullaris syndrome is associated with injury to the sacral cord and lumbar nerve roots. This syndrome is characterized by areflexia in the bladder, bowel, and lower limbs, while the sacral segments occasionally may show preserved reflexes (e.g., bulbocavemosus and micturition reflexes). Cauda equina syndrome is due to injury to the lumbosacral nerve roots in the spinal canal, leading to areflexic bladder, bowel, and lower limbs.

Neurogenic shock can result from SCI. C. H. Tator, Brain Pathology 5:407-413 (1995). Neurogenic shock refers to the hemodynamic triad of hypotension, bradycardia, and peripheral vasodilation resulting from autonomic dysfunction and the interruption of sympathetic nervous system control in acute SCI, and is differentiated from spinal and hypovolemic shock. Hypovolemic shock tends to be associated with tachycardia. Spinal shock is defined as the complete loss of all neurologic function, including reflexes and rectal tone, below a specific level that is associated with autonomic dysfunction. An initial increase in blood pressure is noted due to the release of catecholamines, followed by hypotension. Flaccid paralysis, including of the bowel and bladder, is observed, and sometimes sustained priapism develops. These symptoms tend to last several hours to days until the reflex arcs below the level of the injury begin to function again.

Current therapy for SCI aims to improve motor function and sensation in patients with the disorder. At present, there are no agents that are consistently effective in treating the disorder. Corticosteroids are the mainstay of therapy. Glucocorticoids such as methylprednisolone are thought to reduce the secondary effects of acute SCI, and the use of high-dose methylprednisolone in nonpenetrating acute SCI has become the standard of care in North America. However, the validities of the results are questionable. Nesathurai S. et al., J Trauma 1998 December; 45(6): 1088-93. Therefore, new methods and compounds that are able to treat SCI and related syndromes are needed.

2.2. C-JUN N-Terminal Kinase (JNK)

Three JNK enzymes have been identified. These represent alternatively spliced forms of three different genes: JNK1, JNK2, and JNK3 (Hibi M., Lin A., Smeal T., Minden A., Karin M. Genes Dev. 7:2135-2148, 1993; Mohit A. A., Martin M. H., and Miller C. A. Neuron 14:67-78, 1995; Gupta, S., Barrett, T., Whitmarsh, A. J., Cavanagh, J., Sluss, H. K., Derijard, B. and Davis, R. J. The EMBO J. 15:2760-2770, 1996).

Activation of the JNK pathway has been documented in a number of disease settings, providing the rationale for targeting this pathway for drug discovery. For example, autoimmune and inflammatory diseases arise from the over-activation of the immune system. Activated immune cells express many genes encoding inflammatory molecules, including cytokines, growth factors, cell surface receptors, cell adhesion molecules and degradative enzymes. Many of these genes are regulated by the JNK pathway, through activation of the transcription factors AP-1 and ATF-2, including TNFα, IL-2, E-selectin and matrix metalloproteinases such as collagenase-1 (Manning A. M. and Mercurio F. Exp. Opin Invest. Drugs 6: 555-567, 1997). In addition, molecular genetic approaches have validated the pathogenic role of the JNK pathway in several diseases.

### 3. SUMMARY OF THE INVENTION

This invention encompasses methods of treating and preventing central nervous system (CNS) injury/damage and related syndromes which comprise administering to a patient in need of such treatment or prevention a therapeutically or prophylactically effective amount of a JNK inhibitor, or a pharmaceutically acceptable salt, solvate, hydrate, stereoisomer, clathrate, or prodrug thereof. CNS injury/damage and related syndromes include, but are not limited to, primary brain injury, secondary brain injury, traumatic brain injury, focal brain injury, diffuse axonal injury, head injury, concussion, post-concussion syndrome, cerebral contusion and laceration, subdural hematoma, epidermal hematoma, post-traumatic epilepsy, chronic vegetative state, complete SCI, incomplete SCI, acute SCI, subacute SCI, chronic SCI, central cord syndrome, Brown-Sequard syndrome, anterior cord syndrome, conus medullaris syndrome, cauda equina syndrome, neurogenic shock, spinal shock, altered level of consciousness, headache, nausea, emesis, memory loss, dizziness, diplopia, blurred vision, emotional lability, sleep disturbances, irritability, inability to concentrate, nervousness, behavioral impairment, cognitive deficit, and seizure.

The invention also encompasses methods of managing CNS injury/damage and related syndromes (e.g., lengthening the time of remission of their symptoms) which comprise administering to a patient in need of such management a prophylactically effective amount of a JNK inhibitor, or a pharmaceutically acceptable salt, solvate, hydrate, stereoisomer, clathrate, or prodrug thereof. Each of these methods includes specific dosing or dosing regimens.

The invention further encompasses pharmaceutical compositions, single unit dosage forms, and kits suitable for use in treating, preventing and/or managing CNS injury/damage and related syndromes, which comprise one or more JNK inhibitors, or a pharmaceutically acceptable salt, solvate, hydrate, stereoisomer, clathrate, or prodrug thereof.

The JNK inhibitors, or compounds of the invention, which are described in detail below, are small organic molecules, i.e., having a molecule weight less than 1,000 g/mol. The compounds preferably inhibit JNK activity and TNF-α production.

In particular embodiments of the invention, a JNK inhibitor is used, administered, or formulated with one or more second active agents to treat, prevent or manage CNS injury/damage or related syndromes. In a particular embodiment, the second active agent is useful for treating, preventing or managing CNS injury/damage or related syndromes. Examples of the second active agents include but are not limited to anti-inflammatory agents including nonsteroidal anti-inflammatory drugs (NSAIDs) and steroids, cAMP analogs, diuretics, barbiturates, immunomodulatory agents, immunosuppressive agents, antihypertensives, anticonvulsants, fibrinolytic agents, antipsychotics, antidepressants, benzodiazepines, buspirone, stimulants, amantadine, an IMiD®, a SelCID®, and other standard therapies used for CNS injury/damage and related syndromes.

### 4. DETAILED DESCRIPTION OF THE INVENTION

A first embodiment of the invention encompasses methods of treating or preventing CNS injury/damage and related syndromes, which comprise administering to a patient in need of such treatment or prevention a therapeutically or prophylactically effective amount of a JNK inhibitor, or a pharmaceutically acceptable salt, solvate, hydrate, stereoisomer, clathrate, or prodrug thereof. CNS injury/damage and related syndromes, include, but are not limited to, primary brain injury, secondary brain injury, traumatic brain injury, focal brain injury, diffuse axonal injury, head injury, concussion, post-concussion syndrome, cerebral contusion and laceration, subdural hematoma, epidermal hematoma, post-traumatic epilepsy, chronic vegetative state, complete SCI, incomplete SCI, acute SCI, subacute SCI, chronic SCI, central cord syndrome, Brown-Sequard syndrome, anterior cord syndrome, conus medullaris syndrome, cauda equina syndrome, neurogenic shock, spinal shock, altered level of consciousness, headache, nausea, emesis, memory loss, dizziness, diplopia, blurred vision, emotional lability, sleep disturbances, irritability, inability to concentrate, nervousness, behavioral impairment, cognitive deficit, and seizure.

Another embodiment of the invention encompasses methods of managing CNS injury/damage and related syndromes, which comprise administering to a patient in need of such management a prophylactically effective amount of a JNK inhibitor, or a pharmaceutically acceptable salt, solvate, hydrate, stereoisomer, clathrate, or prodrug thereof.

Another embodiment of the invention encompasses a method of treating, preventing and/or managing CNS injury/damage and related syndromes, which comprises administering to a patient in need of such treatment, prevention and/or management a therapeutically or prophylactically effective amount of a JNK inhibitor, or a pharmaceutically acceptable salt, solvate, hydrate, stereoisomer, clathrate, or prodrug thereof, and a therapeutically or prophylactically effective amount of a second active agent. Without being limited by theory, it is believed that certain JNK inhibitors and agents conventionally used in CNS injury/damage and related syndromes can act in complementary or synergistic ways in the treatment or management of the disorders. It is also believed that the combined use of such agents may reduce or eliminate adverse effects associated with some JNK inhibitors, thereby allowing the administration of larger amounts of JNK inhibitors to patients and/or increasing patient compliance. It is further believed that some JNK inhibitors may reduce or eliminate adverse effects associated with some conventional agents, thereby allowing the administration of larger amounts of the agents to patients and/or increasing patient compliance.

Another embodiment of the invention encompasses a method of reversing, reducing or avoiding an adverse effect associated with the administration of conventional therapy for CNS injury/damage and related syndromes to a patient suffering from CNS injury/damage or a related disorder, which comprises administering to a patient in need of such reversion, reduction or avoidance a therapeutically or prophylactically effective amount of a JNK inhibitor, or a pharmaceutically acceptable salt, solvate, hydrate, stereoisomer, clathrate, or prodrug thereof.

Yet another embodiment of the invention encompasses a pharmaceutical composition comprising a JNK inhibitor, or a pharmaceutically acceptable salt, solvate, hydrate, stereoisomer, clathrate, or prodrug thereof, and a pharmaceutically acceptable carrier, diluent or excipient, wherein the composition is adapted for parenteral or oral administration, and the amount is sufficient to treat or prevent CNS injury/damage and related syndromes, or to ameliorate the symptoms or progress of the syndromes.

Also encompassed by the invention are single unit dosage forms comprising a JNK inhibitor, or a pharmaceutically acceptable salt, solvate, hydrate, stereoisomer, clathrate, or prodrug thereof.

The invention also encompasses kits which comprise a JNK inhibitor, or a pharmaceutically acceptable salt, solvate, hydrate, stereoisomer, clathrate, or prodrug thereof, and a second active agent. The examples of the second active agent include, but are not limited to, anti-inflammatory agents including nonsteroidal anti-inflammatory drugs (NSAIDs) and steroids such as glucocorticoids, cAMP analogs, diuretics, barbiturates, immunomodulatory agents, immunosuppressive agents, antihypertensives, anticonvulsants, fibrinolytic agents, antipsychotics, antidepressants, benzodiazepines, buspirone, stimulants, amantadine, and other known or conventional agents used in patients with CNS injury/damage and related syndromes.

4.1. Definitions

As used herein, the term “patient” means an animal (e.g., cow, horse, sheep, pig, chicken, turkey, quail, cat, dog, mouse, rat, rabbit or guinea pig), preferably a mammal such as a non-primate or a primate (e.g., monkey and human), most preferably a human.

“Alkyl” means a saturated straight chain or branched non-cyclic hydrocarbon having from 1 to 10 carbon atoms. “Lower alkyl” means alkyl, as defined above, having from 1 to 4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

An “alkenyl group” or “alkylidene” mean a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. An alkenyl group can be unsubstituted or substituted. A “cyclic alkylidene” is a ring having from 3 to 8 carbon atoms and including at least one carbon-carbon double bond, wherein the ring can have from 1 to 3 heteroatoms.

An “alkynyl group” means a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. An alkynyl group can be unsubstituted or substituted.

The terms “Halogen” and “Halo” mean fluorine, chlorine, bromine or iodine.

“Haloalkyl” means an alkyl group, wherein alkyl is defined above, substituted with one or more halogen atoms.

“Keto” means a carbonyl group (i.e., C═O).

“Acyl” means an —C(O)alkyl group, wherein alkyl is defined above, including —C(O)CH₃, —C(O)CH₂CH₃, —C(O)(CH₂)₂CH₃, —C(O)(CH₂)₃CH₃, —C(O)(CH₂)₄CH₃, —C(O)(CH₂)₅CH₃, and the like.

“Acyloxy” means an —OC(O)alkyl group, wherein alkyl is defined above, including —OC(O)CH₃, —OC(O)CH₂CH₃, —OC(O)(CH₂)₂CH₃, —OC(O)(CH₂)₃CH₃, —OC(O)(CH₂)₄CH₃, —OC(O)(CH₂)₅CH₃, and the like.

“Ester” means and —C(O)Oalkyl group, wherein alkyl is defined above, including —C(O)OCH₃, —C(O)OCH₂CH₃, —C(O)O(CH₂)₂CH₃, —C(O)O(CH₂)₃CH₃, —C(O)O(CH₂)₄CH₃, —C(O)O(CH₂)₅CH₃, and the like.

“Alkoxy” means —O-(alkyl), wherein alkyl is defined above, including —OCH₃, —OCH₂CH₃, —O(CH₂)₂CH₃, —O(CH₂)₃CH₃, —O(CH₂)₄CH₃, —O(CH₂)₅CH₃, and the like. “Lower alkoxy” means —O-(lower alkyl), wherein lower alkyl is as described above.

“Alkoxyalkoxy” means —O-(alkyl)-O-(alkyl), wherein each alkyl is independently an alkyl group defined above, including —OCH₂OCH₃, —OCH₂CH₂OCH₃, —OCH₂CH₂OCH₂CH₃, and the like.

“Alkoxycarbonyl” means —C(═O)O-(alkyl), wherein alkyl is defined above, including —C(═O)O—CH₃, —C(═O)O—CH₂CH₃, —C(═O)O—(CH₂)₂CH₃, —C(═O)O—(CH₂)₃CH₃, —C(═O)O—(CH₂)₄CH₃, —C(═O)O—(CH₂)₅CH₃, and the like.

“Alkoxycarbonylalkyl” means -(alkyl)-C(═O)O-(alkyl), wherein each alkyl is independently defined above, including —CH₂—C(═O)O—CH₃, —CH₂—C(═O)O—CH₂CH₃, —CH₂—C(═O)O—(CH₂)₂CH₃, —CH₂—C(═O)O—(CH₂)₃CH₃, —CH₂—C(═O)O—(CH₂)₄CH₃, —CH₂—C(═O)O—(CH₂)₅CH₃, and the like.

“Alkoxyalkyl” means -(alkyl)-O-(alkyl), wherein each alkyl is independently an alkyl group defined above, including —CH₂OCH₃, —CH₂OCH₂CH₃, —(CH₂)₂OCH₂CH₃, —(CH₂)₂O(CH₂)₂CH₃, and the like.

“Aryl” means a carbocyclic aromatic group containing from 5 to 10 ring atoms. Representative examples include, but are not limited to, phenyl, tolyl, anthracenyl, fluorenyl, indenyl, azulenyl, pyridinyl and naphthyl, as well as benzo-fused carbocyclic moieties including 5,6,7,8-tetrahydronaphthyl. A carbocyclic aromatic group can be unsubstituted or substituted. In one embodiment, the carbocyclic aromatic group is a phenyl group.

“Aryloxy” means —O-aryl group, wherein aryl is as defined above. An aryloxy group can be unsubstituted or substituted. In one embodiment, the aryl ring of an aryloxy group is a phenyl group.

“Arylalkyl” means -(alkyl)-(aryl), wherein alkyl and aryl are as defined above, including —(CH₂)phenyl, —(CH₂)₂phenyl, —(CH₂)₃phenyl, —CH(phenyl)₂, —CH(phenyl)₃, —(CH₂)tolyl, —(CH₂)anthracenyl, —(CH₂)fluorenyl, —(CH₂)indenyl, —(CH₂)azulenyl, —(CH₂)pyridinyl, —(CH₂)naphthyl, and the like.

“Arylalkyloxy” means —O-(alkyl)-(aryl), wherein alkyl and aryl are defined above, including —O—(CH₂)₂phenyl, —O—(CH₂)₃phenyl, —O—CH(phenyl)₂, —O—CH(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)pyridinyl, —O—(CH₂)naphthyl, and the like.

“Aryloxyalkyl” means -(alkyl)-O-(aryl), wherein alkyl and aryl are defined above, including —CH₂—O-(phenyl), —(CH₂)₂—O-phenyl, —(CH₂)₃—O-phenyl, —(CH₂)—O-tolyl, —(CH₂)—O-anthracenyl, —(CH₂)—O-fluorenyl, —(CH₂)—O-indenyl, —(CH₂)—O-azulenyl, —(CH₂)—O-pyridinyl, —(CH₂)—O-naphthyl, and the like.

“Cycloalkyl” means a monocyclic or polycyclic saturated ring having carbon and hydrogen atoms and having no carbon-carbon multiple bonds. Examples of cycloalkyl groups include, but are not limited to, (C₃-C₇)cycloalkyl groups, including cyclopropyl, cyclobutyl, cyclopentyl, cyclohexyl, and cycloheptyl, and saturated cyclic and bicyclic terpenes. A cycloalkyl group can be unsubstituted or substituted. In one embodiment, the cycloalkyl group is a monocyclic ring or bicyclic ring.

“Cycloalkyloxy” means —O-(cycloalkyl), wherein cycloalkyl is defined above, including —O-cyclopropyl, —O-cyclobutyl, —O-cyclopentyl, —O-cyclohexyl, —O-cycloheptyl and the like.

“Cycloalkylalkyloxy” means —O-(alkyl)-(cycloalkyl), wherein cycloalkyl and alkyl are defined above, including —O—CH₂-cyclopropyl, —O—(CH₂)₂-cyclopropyl, —O—(CH₂)₃-cyclopropyl, —O—(CH₂)₄-cyclopropyl, O—CH₂-cyclobutyl, O—CH₂-cyclopentyl, O—CH₂-cyclohexyl, O—CH₂-cycloheptyl, and the like.

“Aminoalkoxy” means —O-(alkyl)-NH₂, wherein alkyl is defined above, such as —O—CH₂—NH₂, —O—(CH₂)₂—NH₂, —O—(CH₂)₃—NH₂, —O—(CH₂)₄—NH₂, —O—(CH₂)₅—NH₂, and the like.

“Mono-alkylamino” means —NH(alkyl), wherein alkyl is defined above, such as —NHCH₃, —NHCH₂CH₃, —NH(CH₂)₂CH₃, —NH(CH₂)₃CH₃, —NH(CH₂)₄CH₃, —NH(CH₂)₅CH₃, and the like.

“Di-alkylamino” means —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃), and the like.

“Mono-alkylaminoalkoxy” means —O-(alkyl)-NH(alkyl), wherein each alkyl is independently an alkyl group defined above, including —O—(CH₂)—NHCH₃, —O—(CH₂)—NHCH₂CH₃, —O—(CH₂)—NH(CH₂)₂CH₃, —O—(CH₂)—NH(CH₂)₃CH₃, —O—(CH₂)—NH(CH₂)₄CH₃, —O—(CH₂)—NH(CH₂)₅CH₃, —O—(CH₂)₂—NHCH₃, and the like.

“Di-alkylaminoalkoxy” means —O-(alkyl)-N(alkyl)(alkyl), wherein each alkyl is independently an alkyl group defined above, including —O—(CH₂)—N(CH₃)₂, —O—(CH₂)—N(CH₂CH₃)₂, —O—(CH₂)—N((CH₂)₂CH₃)₂, —O—(CH₂)—N(CH₃)(CH₂CH₃), and the like.

“Arylamino” means —NH(aryl), wherein aryl is defined above, including —NH(phenyl), —NH(tolyl), —NH(anthracenyl), —NH(fluorenyl), —NH(indenyl), —NH(azulenyl), —NH(pyridinyl), —NH(naphthyl), and the like.

“Arylalkylamino” means —NH-(alkyl)-(aryl), wherein alkyl and aryl are defined above, including —NH—CH₂-(phenyl), —NH—CH₂-(tolyl), —NH—CH₂-(anthracenyl), —NH—CH₂-(fluorenyl), —NH—CH₂-(indenyl), —NH—CH₂-(azulenyl), —NH—CH₂-(pyridinyl), —NH—CH₂-(naphthyl), —NH—(CH₂)₂-(phenyl) and the like.

“Alkylamino” means mono-alkylamino or di-alkylamino as defined above, such as —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and the like.

“Cycloalkylamino” means —NH-(cycloalkyl), wherein cycloalkyl is as defined above, including —NH-cyclopropyl, —NH-cyclobutyl, —NH-cyclopentyl, —NH-cyclohexyl, —NH-cycloheptyl, and the like.

“Carboxyl” and “carboxy” mean —COOH.

“Cycloalkylalkylamino” means —NH-(alkyl)-(cycloalkyl), wherein alkyl and cycloalkyl are defined above, including —NH—CH₂-cyclopropyl, —NH—CH₂-cyclobutyl, —NH—CH₂-cyclopentyl, —NH—CH₂-cyclohexyl, —NH—CH₂-cycloheptyl, —NH—(CH₂)₂-cyclopropyl and the like.

“Aminoalkyl” means -(alkyl)-NH₂, wherein alkyl is defined above, including CH₂—NH₂, —(CH₂)₂—NH₂, —(CH₂)₃—NH₂, —(CH₂)₄—NH₂, —(CH₂)₅—NH₂ and the like.

“Mono-alkylaminoalkyl” means -(alkyl)-NH(alkyl), wherein each alkyl is independently an alkyl group defined above, including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂—NH(CH₂)₅CH₃, —(CH₂)₂—NH—CH₃, and the like.

“Di-alkylaminoalkyl” means -(alkyl)-N(alkyl)(alkyl), wherein each alkyl is independently an alkyl group defined above, including —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, thietanyl and oxazolyl.

“Heteroarylalkyl” means -(alkyl)-(heteroaryl), wherein alkyl and heteroaryl are defined above, including —CH₂-triazolyl, —CH₂-tetrazolyl, —CH₂-oxadiazolyl, —CH₂-pyridyl, —CH₂-furyl, —CH₂-benzofuranyl, —CH₂-thiophenyl, —CH₂-benzothiophenyl, —CH₂-quinolinyl, —CH₂-pyrrolyl, —CH₂-indolyl, —CH₂-oxazolyl, —CH₂-benzoxazolyl, —CH₂-imidazolyl, —CH₂-benzimidazolyl, —CH₂-thiazolyl, —CH₂-benzothiazolyl, —CH₂-isoxazolyl, —CH₂-pyrazolyl, —CH₂-isothiazolyl, —CH₂-pyridazinyl, —CH₂-pyrimidinyl, —CH₂-pyrazinyl, —CH₂-triazinyl, —CH₂-cinnolinyl, —CH₂-phthalazinyl, —CH₂-quinazolinyl, —CH₂-pyrimidyl, —CH₂-oxetanyl, —CH₂-azepinyl, —CH₂-piperazinyl, —CH₂-morpholinyl, —CH₂-dioxanyl, —CH₂-thietanyl, —CH₂-oxazolyl, —(CH₂)₂-triazolyl, and the like.

“Heterocycle” means a 5- to 7-membered monocyclic, or 7- to 10-membered bicyclic, heterocyclic ring which is either saturated, unsaturated, and which contains from 1 to 4 heteroatoms independently selected from nitrogen, oxygen and sulfur, and wherein the nitrogen and sulfur heteroatoms can be optionally oxidized, and the nitrogen heteroatom can be optionally quaternized, including bicyclic rings in which any of the above heterocycles are fused to a benzene ring. The heterocycle can be attached via any heteroatom or carbon atom. Heterocycles include heteroaryls as defined above. Representative heterocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocycle fused to phenyl” means a heterocycle, wherein heterocycle is defined as above, that is attached to a phenyl ring at two adjacent carbon atoms of the phenyl ring.

“Heterocycloalkyl” means -(alkyl)-(heterocycle), wherein alkyl and heterocycle are defined above, including —CH₂-morpholinyl, —CH₂-pyrrolidinonyl, —CH₂-pyrrolidinyl, —CH₂-piperidinyl, —CH₂-hydantoinyl, —CH₂-valerolactamyl, —CH₂-oxiranyl, —CH₂-oxetanyl, —CH₂-tetrahydrofuranyl, —CH₂-tetrahydropyranyl, —CH₂-tetrahydropyridinyl, —CH₂-tetrahydroprimidinyl, —CH₂-tetrahydrothiophenyl, —CH₂-tetrahydrothiopyranyl, —CH₂-tetrahydropyrimidinyl, —CH₂-tetrahydrothiophenyl, —CH₂-tetrahydrothiopyranyl, and the like.

The term “substituted” as used herein means any of the above groups (i.e., aryl, arylalkyl, heterocycle and heterocycloalkyl) wherein at least one hydrogen atom of the moiety being substituted is replaced with a substituent. In one embodiment, each carbon atom of the group being substituted is substituted with no more that two substituents. In another embodiment, each carbon atom of the group being substituted is substituted with no more than one substituent. In the case of a keto substituent, two hydrogen atoms are replaced with an oxygen which is attached to the carbon via a double bond. Substituents include halogen, hydroxyl, alkyl, haloalkyl, mono- or di-substituted aminoalkyl, alkyloxyalkyl, aryl, arylalkyl, heterocycle, heterocycloalkyl, —NRₐR_{b}, —NRₐC(═O)R_{b}, —NRₐC(═O)NRₐR_{b}, —NRₐC(═O)OR_{b} —NRₐSO₂R_{b}, —ORₐ, C(═O)Rₐ C(═O)ORₐ —C(═O)NRₐR_{b}, —OC(═O)Rₐ, —OC(═O)ORₐ, —OC(═O)NRₐR_{b}, —NRₐSO₂R_{b}, or a radical of the formula —Y-Z-Rₐ where Y is alkanediyl, or a direct bond, Z is —O—, —S—, —N(R_{b})—, —C(═O)—, —C(═O)O—, —OC(═O)—, —N(R_{b})C(═O)—, —C(═O)N(R_{b})— or a direct bond, wherein Rₐ and R_{b} are the same or different and independently hydrogen, amino, alkyl, haloalkyl, aryl, arylalkyl, heterocycle, or heterocylealkyl, or wherein Rₐ and R_{b} taken together with the nitrogen atom to which they are attached form a heterocycle.

“Haloalkyl” means alkyl, wherein alkyl is defined as above, having one or more hydrogen atoms replaced with halogen, wherein halogen is as defined above, including —CF₃, —CHF₂, —CH₂F, —CBr₃, —CHBr₂, —CH₂Br, —CCl₃, —CHCl₂, —CH₂Cl, —CI₃, —CHI₂, —CH₂I, —CH₂—CF₃, —CH₂—CHF₂, —CH₂—CH₂F, —CH₂—CBr₃, —CH₂—CHBr₂, —CH₂—CH₂Br, —CH₂—CCl₃, —CH₂—CHCl₂, —CH₂—CH₂Cl, —CH₂—CI₃, —CH₂—CHI₂, —CH₂—CH₂I, and the like.

“Hydroxyalkyl” means alkyl, wherein alkyl is as defined above, having one or more hydrogen atoms replaced with hydroxy, including —CH₂OH, —CH₂CH₂OH, —(CH₂)₂CH₂OH, —(CH₂)₃CH₂OH, —(CH₂)₄CH₂OH, —(CH₂)₅CH₂OH, —CH(OH)—CH₃, —CH₂CH(OH)CH₃, and the like.

“Hydroxy” means —OH.

“Sulfonyl” means —SO₃H.

“Sulfonylalkyl” means —SO₂-(alkyl), wherein alkyl is defined above, including —SO₂—CH₃, —SO₂—CH₂CH₃, —SO₂—(CH₂)₂CH₃, —SO₂—(CH₂)₃CH₃, —SO₂—(CH₂)₄CH₃, —SO₂—(CH₂)₅CH₃, and the like.

“Sulfinylalkyl” means —SO-(alkyl), wherein alkyl is defined above, including —SO—CH₃, —SO—CH₂CH₃, —SO—(CH₂)₂CH₃, —SO—(CH₂)₃CH₃, —SO—(CH₂)₄CH₃, —SO—(CH₂)₅CH₃, and the like.

“Sulfonamidoalkyl” means —NHSO₂-(alkyl), wherein aklyl is defined above, including —NHSO₂—CH₃, —NHSO₂—CH₂CH₃, —NHSO₂—(CH₂)₂CH₃, —NHSO₂—(CH₂)₃CH₃, —NHSO₂—(CH₂)₄CH₃, —NHSO₂—(CH₂)₅CH₃, and the like.

“Thioalkyl” means —S-(alkyl), wherein alkyl is defined above, including —S—CH₃, —S—CH₂CH₃, —S—(CH₂)₂CH₃, —S—(CH₂)₃CH₃, —S—(CH₂)₄CH₃, —S—(CH₂)₅CH₃, and the like.

As used herein, the term “JNK inhibitor(s)” encompasses, but is not limited to, compounds disclosed herein. Without being limited by theory, specific JNK inhibitors capable of inhibiting the activity of JNK in vitro or in vivo. The JNK inhibitor can be in the form of a pharmaceutically acceptable salt, free base, solvate, hydrate, stereoisomer, clathrate or prodrug thereof. Such inhibitory activity can be determined by an assay or animal model well-known in the art including those set forth in Section 5. In one embodiment, the JNK inhibitor is a compound of structure (I)-(III).

“JNK” means a protein or an isoform thereof expressed by a JNK 1, JNK 2, or JNK 3 gene (Gupta, S., Barrett, T., Whitmarsh, A. J., Cavanagh, J., Sluss, H. K., Derijard, B. and Davis, R. J. The EMBO J. 15:2760-2770 (1996)).

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases known in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of JNK inhibitors that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of a JNK inhibitor that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” and “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyloxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

Various JNK inhibitors contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of JNK inhibitors may be used in methods and compositions of the invention. The purified (R) or (S) enantiomers of the specific compounds disclosed herein may be used substantially free of its other enantiomer.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound.

As used herein and unless otherwise indicated, the term “enantiomerically pure” means a stereomerically pure composition of a compound having one chiral center. Similarly, the term “enantiomerically enriched” means a stereomerically enriched composition of a compound having one chiral center.

4.2. JNK Inhibitors

Compounds used in the invention include racemic, stereomerically pure and stereomerically enriched JNK inhibitor, stereomerically and enantiomerically pure compounds that have selective JNK inhibitory activities, and pharmaceutically acceptable salts, solvates, hydrates, stereoisomers, clathrates, and prodrugs thereof.

Compounds of the invention can either be commercially purchased or prepared according to the methods described in the patents or patent publications disclosed herein. Further, optically pure compositions can be asymmetrically synthesized or resolved using known resolving agents or chiral columns as well as other standard synthetic organic chemistry techniques.

In one embodiment, the JNK inhibitor has the following structure (I):

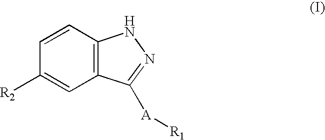

wherein:

A is a direct bond, —(CH₂)ₐ—, —(CH₂)_{b}CH═CH(CH₂)_{c}—, or —(CH₂)_{b}C≡C(CH₂)_{c}—;

R₁ is aryl, heteroaryl or heterocycle fused to phenyl, each being optionally substituted with one to four substituents independently selected from R₃;

R₂ is —R₃, —R₄, —(CH₂)_{b}C(═O)R₅, —(CH₂)_{b}C(═O)OR₅, —(CH₂)_{b}C(═O)NR₅R₆,

—(CH₂)_{b}C(═O)NR₅(CH₂)CC(═O)R₆, —(CH₂)_{b}NR₅C(═O)R₆,

—(CH₂)_{b}NR₅C(═O)NR₆R₇, —(CH₂)_{b}NR₅R₆, —(CH₂)_{b}OR₅,

—(CH₂)_{b}SO_{d}R₅ or —(CH₂)_{b}SO₂NR₅R₆;

a is 1, 2, 3, 4, 5 or 6;

b and c are the same or different and at each occurrence independently selected from 0, 1, 2, 3 or 4;

d is at each occurrence 0, 1 or 2;

R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;

R₄ is alkyl, aryl, arylalkyl, heterocycle or heterocycloalkyl, each being optionally substituted with one to four substituents independently selected from R₃, or R₄ is halogen or hydroxy;

R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and

R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

In one embodiment, -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3 and wherein R₈ and R₉ are defined above.

In another embodiment, R₂ is —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{b}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆, and b is an integer ranging from 0-4.

In another embodiment, R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment, R₂ is 3-triazolyl or 5-tetrazolyl.

In another embodiment:

(a) -A-R₁ is phenyl, optionally substituted with one to four substituents independently selected from halogen, alkoxy, —NR₈C(═O)R₉, —C(═O)NR₈R₉,

and —O(CH₂)_{b}NR₈R₉, wherein b is 2 or 3; and

(b) R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment:

(a) -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is 3-triazolyl or 5-tetrazolyl.

In another embodiment, R₂ is R₄, and R₄ is 3-triazolyl, optionally substituted at its 5-position with:

(a) a C₁-C₄ straight or branched chain alkyl group optionally substituted with a hydroxyl, methylamino, dimethylamino or 1-pyrrolidinyl group; or

(b) a 2-pyrrolidinyl group.

In another embodiment, R₂ is R₄, and R₄ is 3-triazolyl, optionally substituted at its 5-position with: methyl, n-propyl, isopropyl, 1-hydroxyethyl, 3-hydroxypropyl, methylaminomethyl, dimethylaminomethyl, 1-(dimethylamino)ethyl, 1-pyrrolidinylmethyl or 2-pyrrolidinyl.

In another embodiment, the compounds of structure (I) have structure (IA) when A is a direct bond, or have structure (IB) when A is —(CH₂)ₐ—:

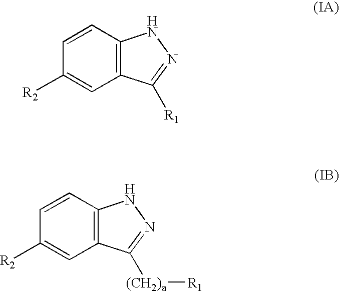

In other embodiments, the compounds of structure (I) have structure (IC) when A is a —(CH₂)_{b}CH═CH(CH₂)C—, and have structure (ID) when A is —(CH₂)_{b}C≡C(CH₂)_{c}—:

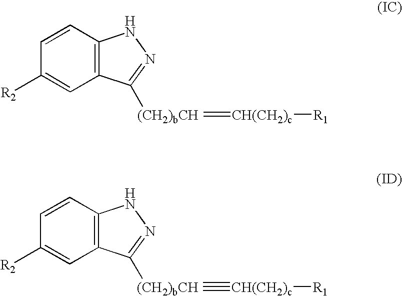

In further embodiments of this invention, R₁ of structure (I) is aryl or substituted aryl, such as phenyl or substituted phenyl as represented by the following structure (IE):

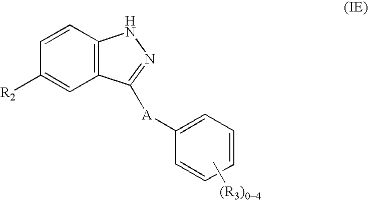

In another embodiment, R₂ of structure (I) is —(CH₂)_{b}NR₄(C═O)R₅. In one aspect of this embodiment, b=0 and the compounds have the following structure (IF):

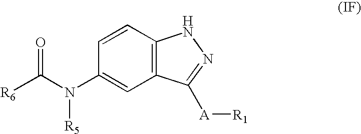

Representative R₂ groups of the compounds of structure (I) include alkyl (such as methyl and ethyl), halo (such as chloro and fluoro), haloalkyl (such as trifluoromethyl), hydroxy, alkoxy (such as methoxy and ethoxy), amino, arylalkyloxy (such as benzyloxy), mono- or di-alkylamine (such as —NHCH₃, —N(CH₃)₂ and —NHCH₂CH₃), —NHC(═O)R₄ wherein R₆ is a substituted or unsubstituted phenyl or heteroaryl (such as phenyl or heteroaryl substituted with hydroxy, carboxy, amino, ester, alkoxy, alkyl, aryl, haloalkyl, halo, —CONH₂ and —CONH alkyl), —NH(heteroarylalkyl) (such as —NHCH₂(3-pyridyl), —NHCH₂(4-pyridyl), heteroaryl (such as pyrazolo, triazolo and tetrazolo), —C(═O)NHR₆ wherein R₆ is hydrogen, alkyl, or as defined above (such as —C(═O)NH₂, —C(═O)NHCH₃, —C(═O)NH(H-carboxyphenyl), —C(═O)N(CH₃)₂), arylalkenyl (such as phenylvinyl, 3-nitrophenylvinyl, 4-carboxyphenylvinyl), heteroarylalkenyl (such as 2-pyridylvinyl, 4-pyridylvinyl).

Representative R₃ groups of the compounds of structure (I) include halogen (such as chloro and fluoro), alkyl (such as methyl, ethyl and isopropyl), haloalkyl (such as trifluoromethyl), hydroxy, alkoxy (such as methoxy, ethoxy, n-propyloxy and isobutyloxy), amino, mono- or di-alkylamino (such as dimethylamine), aryl (such as phenyl), carboxy, nitro, cyano, sulfinylalkyl (such as methylsulfinyl), sulfonylalkyl (such as methylsulfonyl), sulfonamidoalkyl (such as —NHSO₂CH₃), —NR₈C(═O)(CH₂)_{b}OR₉ (such as NHC(═O)CH₂OCH₃), NHC(═O)R₉ (such as —NHC(═O)CH₃, —NHC(═O)CH₂C₆H₅, —NHC(═O)(2-furanyl)), and —O(CH₂)_{b}NR₈R₉ (such as —O(CH₂)₂N(CH₃)₂).

The compounds of structure (I) can be made using organic synthesis techniques known to those skilled in the art, as well as by the methods described in International Publication No. WO 02/10137 (particularly in Examples 1-430, at page 35, line 1 to page 396, line 12), published Feb. 7, 2002, which is incorporated herein by reference in its entirety. Further, specific examples of these compounds are found in this publication.

Illustrative examples of JNK inhibitors of structure (I) are: 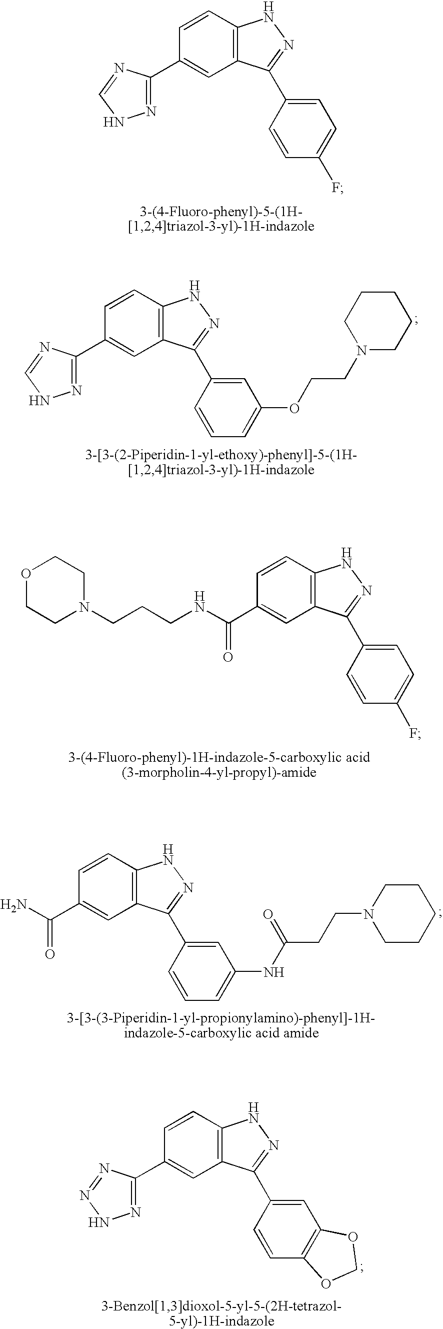 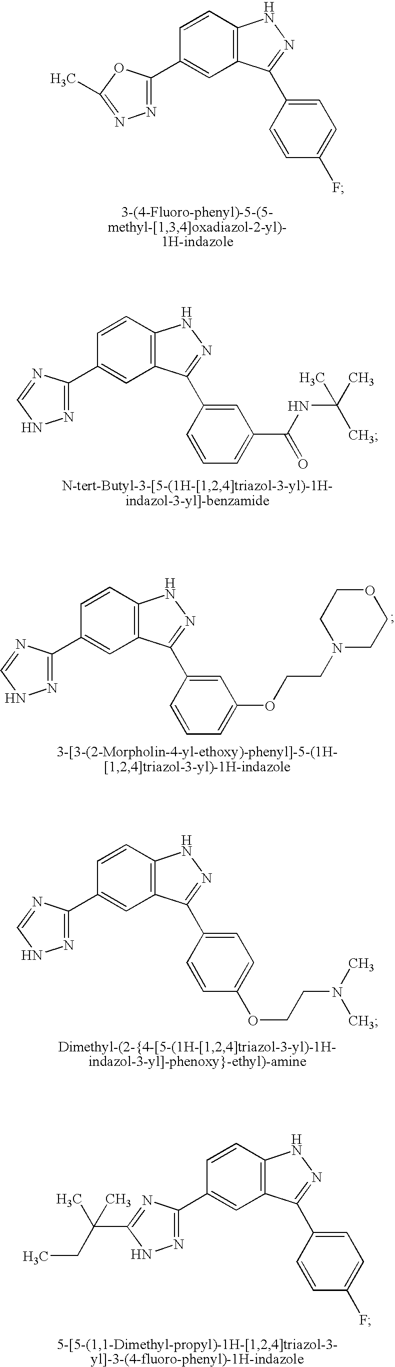 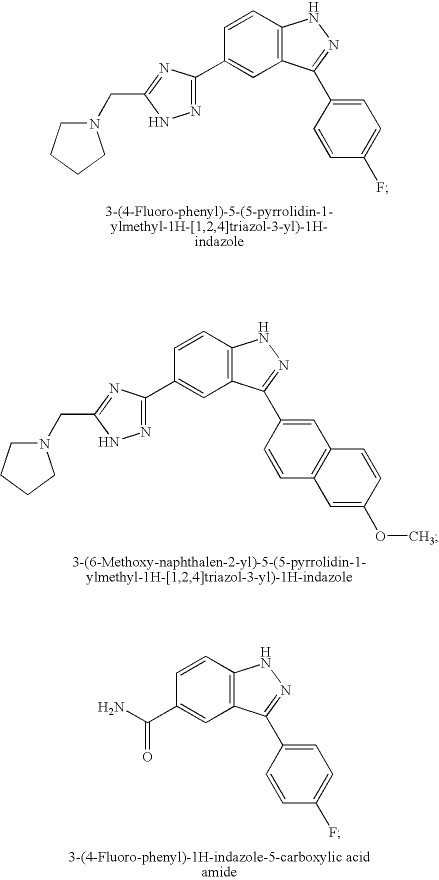
and pharmaceutically acceptable salts thereof.

In another embodiment, the JNK inhibitor has the following structure (II):

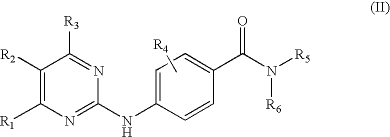

wherein:

R₁ is aryl or heteroaryl optionally substituted with one to four substituents independently selected from R₇;

R₂ is hydrogen;

R₃ is hydrogen or lower alkyl;

R₄ represents one to four optional substituents, wherein each substituent is the same or different and independently selected from halogen, hydroxy, lower alkyl and lower alkoxy;

R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;

or R₅ and R₆ taken together with the nitrogen atom to which they are attached to form a heterocycle or substituted heterocycle;

R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, substituted heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;

R₈, R₉, R₁₀ and R₁ ₁ are the same or different and at each occurrence independently hydrogen, alkyl, aryl, arylalkyl, heterocycle, heterocycloalkyl;

or R₈ and R₉ taken together with the atom or atoms to which they are attached to form a heterocycle;

a and b are the same or different and at each occurrence independently selected from 0, 1, 2, 3 or 4; and

c is at each occurrence 0, 1 or 2.

In one embodiment, R₁ is a substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl or quinazolinyl.

In another embodiment R₁ is substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, preferably phenyl. When R₁ is a substituted aryl, the substituents are defined below. In one embodiment, when substituted, R₁ is substituted with a halogen, —SO₂R₈ or —SO₂R₈R₉.

In another embodiment, R₅ and R₆, taken together with the nitrogen atom to which they are attached form a substituted or unsubstituted nitrogen-containing non-aromatic heterocycle, in one embodiment, piperazinyl, piperidinyl or morpholinyl.

When R₅ and R₆, taken together with the nitrogen atom to which they are attached form substituted piperazinyl, piperadinyl or morpholinyl, the piperazinyl, piperadinyl or morpholinyl is substituted with one or more substituents defined below. In one embodiment, when substituted, the substituent is alkyl, amino, alkylamino, alkoxyalkyl, acyl, pyrrolidinyl or piperidinyl.

In one embodiment, R₃ is hydrogen and R₄ is not present, and the JNK inhibitor has the following structure (IIA): 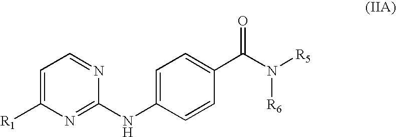
and pharmaceutically acceptable salts thereof.

In a more specific embodiment, R₁ is phenyl optionally substituted with R₇, and having the following structure (IIB): 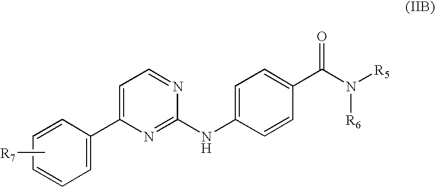
and pharmaceutically acceptable salts thereof.

In still a further embodiment, R₇ is at the para position of the phenyl group relative to the pyrimidine, as represented by the following structure (IIC): 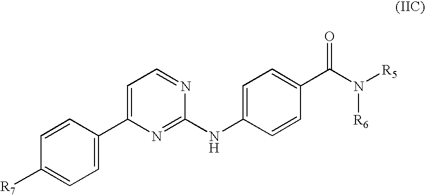
and pharmaceutically acceptable salts thereof.

The JNK inhibitors of structure (II) can be made using organic synthesis techniques known to those skilled in the art, as well as by the methods described in International Publication No. WO 02/46170 (particularly Examples 1-27 at page 23, line 5 to page 183, line 25), published Jun. 13, 2002, which is hereby incorporated by reference in itsr entirety. Further, specific examples of these compounds are found in the publication.

Illustrative examples of JNK inhibitors of structure (II) are: 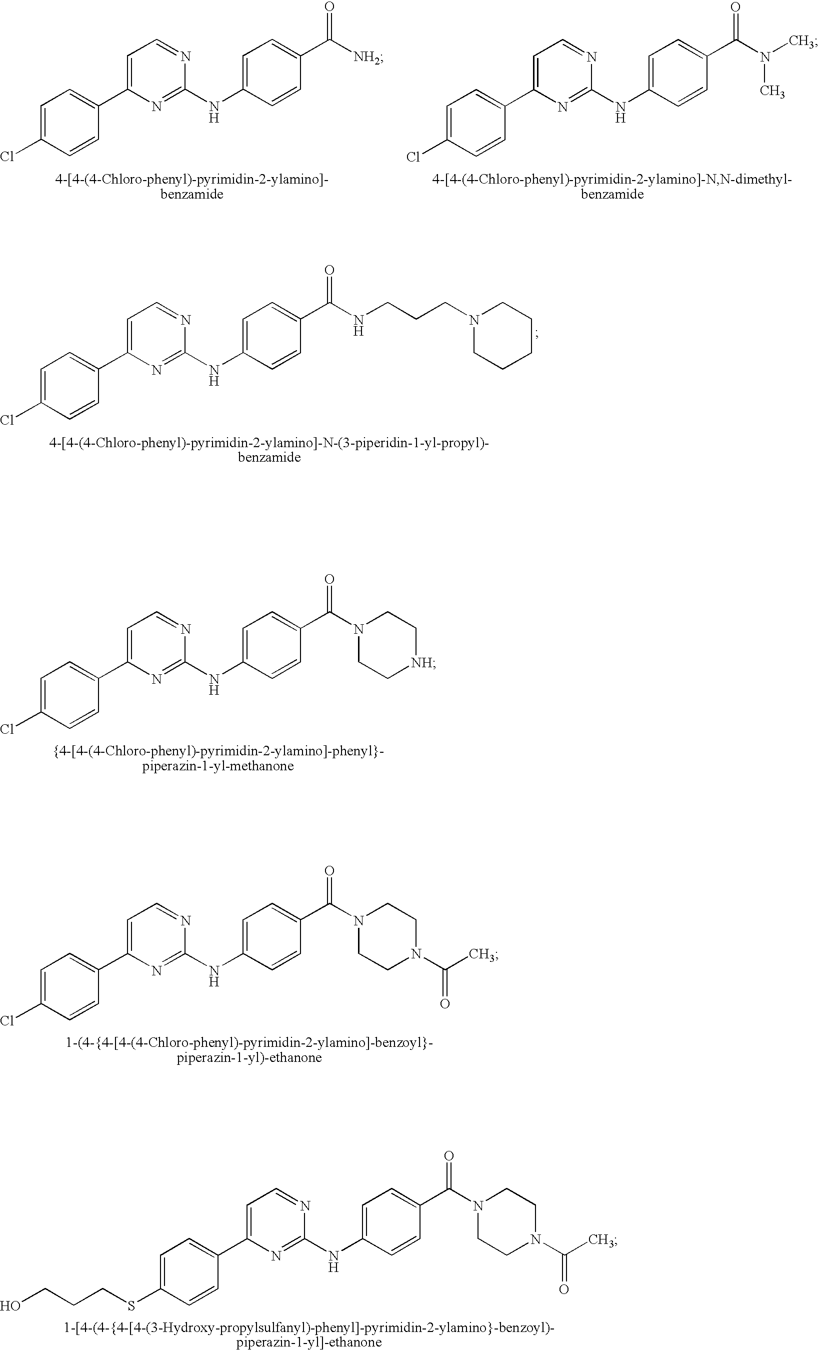 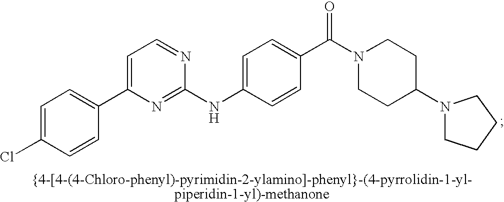
and pharmaceutically acceptable salts thereof.

In another embodiment, the JNK inhibitor has the following structure (III):

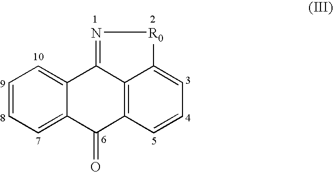

wherein R₀ is —O—, —S—, —S(O)—, —S(O)₂—, NH or —CH₂—;

the compound of structure (III) being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

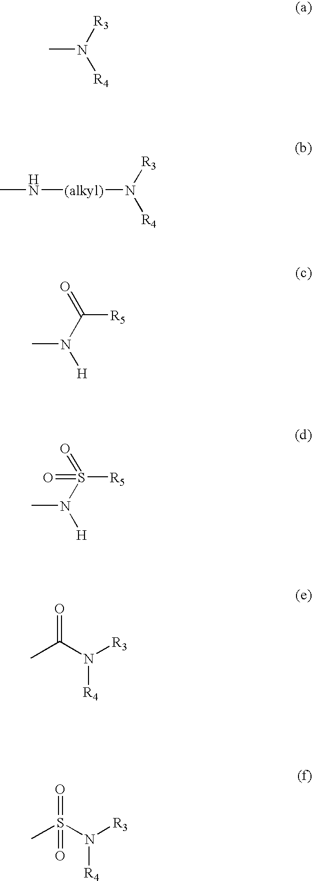

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In another embodiment, the JNK inhibitor has the following structure (IIIA):

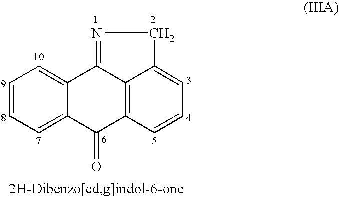

being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

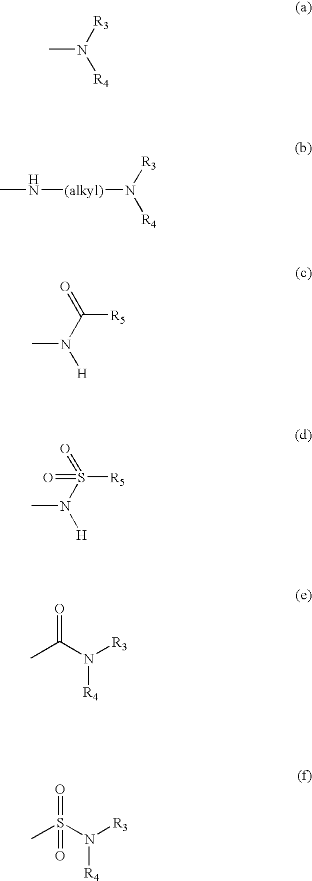

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIA) is that wherein the first or second substituent is present at the 5, 7, or 9 position. In one embodiment, the first or second substituent is present at the 5 or 7 position.

A second subclass of compounds of structure (IIIA) is that wherein the first or second substituent is present at the 5, 7, or 9 position;

the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK inhibitor has the following structure (IIIB):

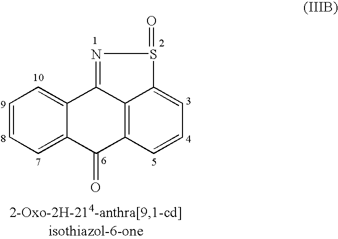

being (i) unsubstituted, (ii) monosubstituted and having a first substituent, or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b) (c), (d), (e), or (f):

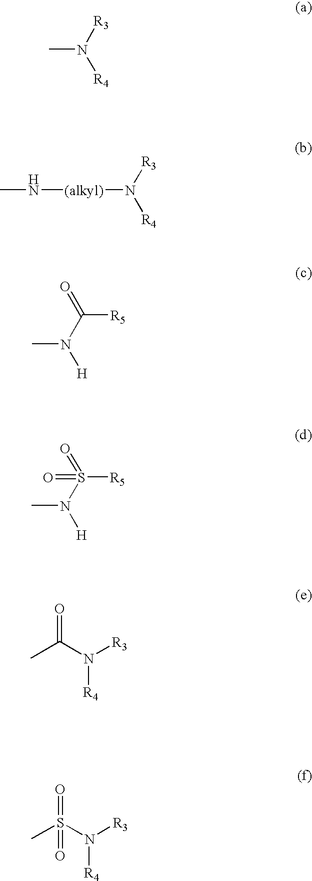

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIB) is that wherein the first or second substituent is present at the 5, 7, or 9 position. In one embodiment, the first or second substituent is present at the 5 or 7 position.

A second subclass of the compounds of structure (IIIB) is that wherein the first or second substituent is independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK inhibitor has the following structure (IIIC):

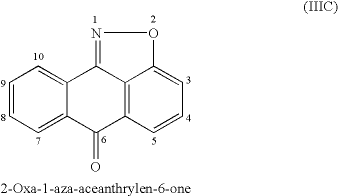

being (i) monosubstituted and having a first substituent or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c) (d), (e), or (f):

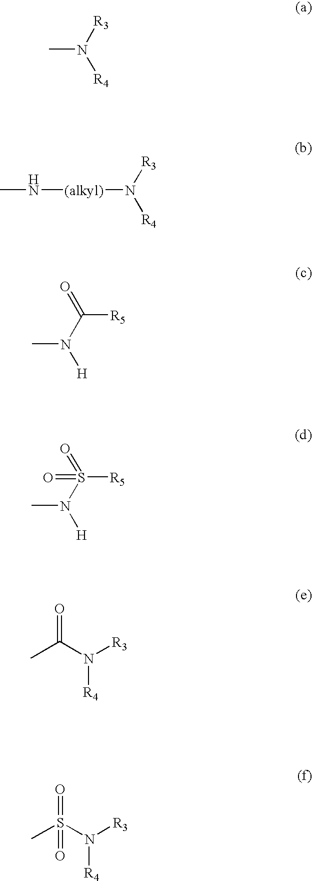

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIC) is that wherein the first or second substituent is present at the 5, 7, or 9 position. In one embodiment, the first or second substituent is present at the 5 or 7 position.

A second subclass of the compounds of structure (IIIC) is that wherein the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK inhibitor has the following structure (IIID):

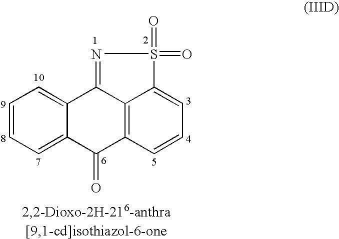

being (i) monosubstituted and having a first substituent present at the 5, 7, or 9 position, (ii) disubstituted and having a first substituent present at the 5 position and a second substituent present at the 7 position, (iii) disubstituted and having a first substituent present at the 5 position and a second substituent present at the 9 position, or (iv) disubstituted and having a first substituent present at the 7 position and a second substituent present at the 9 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

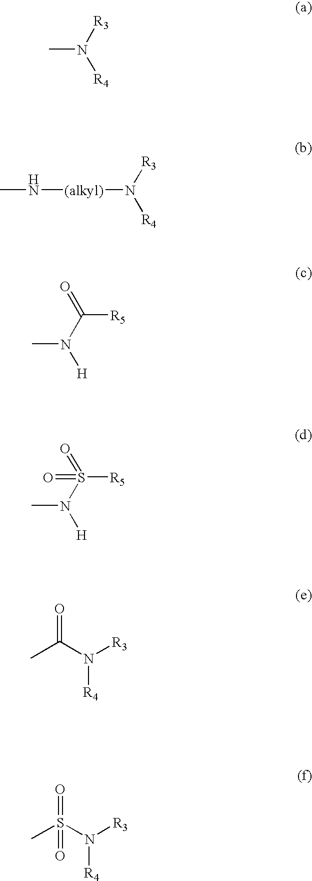

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIID) is that wherein the first or second substituent is present at the 5 or 7 position.

A second subclass of the compounds of structure (IIID) is that wherein the first or second substituent is independently alkyl, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (c), (d), (e), or (f).

Another subclass of the compounds of structure (IIID) is that wherein the first and second substituent are independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, alkoxycarbonyl, or cycloalkylalkyl.

In another embodiment, the JNK inhibitor has the following structure (IIIE):

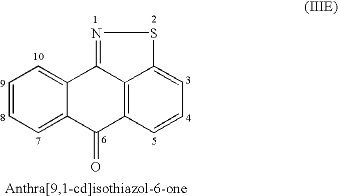

being (i) monosubstituted and having a first substituent present at the 5, 7, or 9 position, (ii) disubstituted and having a first substituent present at the 5 position and a second substituent present at the 9 position, (iii) disubstituted and having a first substituent present at the 7 position and a second substituent present at the 9 position, or (iv) disubstituted and having a first substituent present at the 5 position and a second substituent present at the 7 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

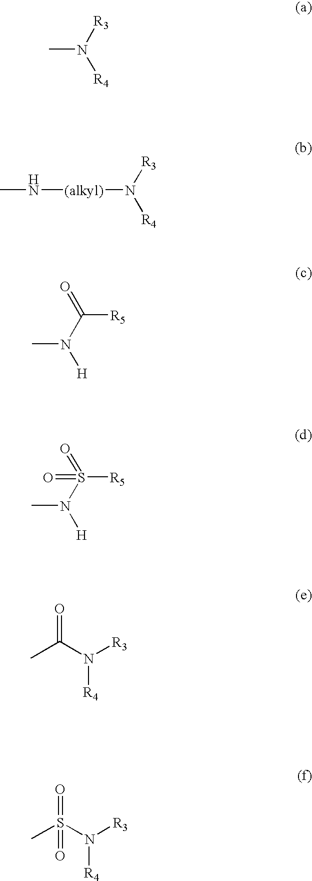

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIE) is that wherein the first or second substituent is present at the 5 or 7 position.

A second subclass of the compounds of structure (IIIE) is that wherein the compound of structure (IIIE) is disubstituted and at least one of the substituents is a group represented by the structure (d) or (f).

Another subclass of the compounds of structure (IIIE) is that wherein the compounds are monosubstituted. Yet another subclass of compounds is that wherein the compounds are monosubstituted at the 5 or 7 position with a group represented by the structure (e) or (f).

In another embodiment, the JNK inhibitor has the following structure (IIIF):

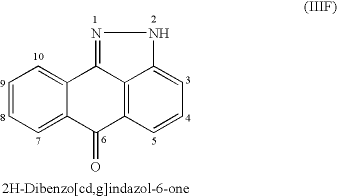

being (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

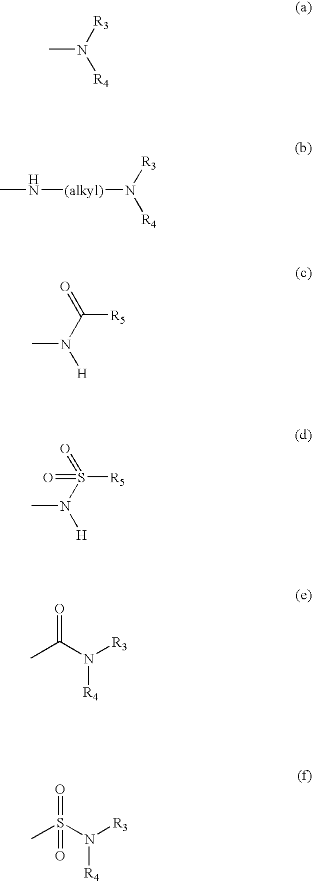

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In one embodiment, the compound of structure (IIIF), or a pharmaceutically acceptable salt thereof is unsubstituted at the 3, 4, 5, 7, 8, 9, or 10 position.

The JNK inhibitors of structure (III) can be made using organic synthesis techniques known to those skilled in the art, as well as by the methods described in International Publication No. WO 01/12609 (particularly Examples 1-7 at page 24, line 6 to page 49, line 16), published Feb. 22, 2001, as well as International Publication No. WO 02/066450 (particularly compounds AA-HG at pages 59-108), published Aug. 29, 2002, each of which is hereby incorporated by reference in its entirety. Further, specific examples of these compounds can be found in the publications.

Illustrative examples of JNK inhibitors of structure (III) are: 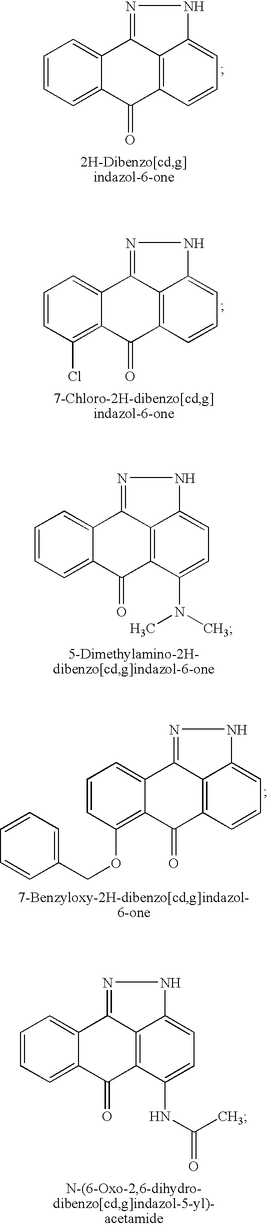 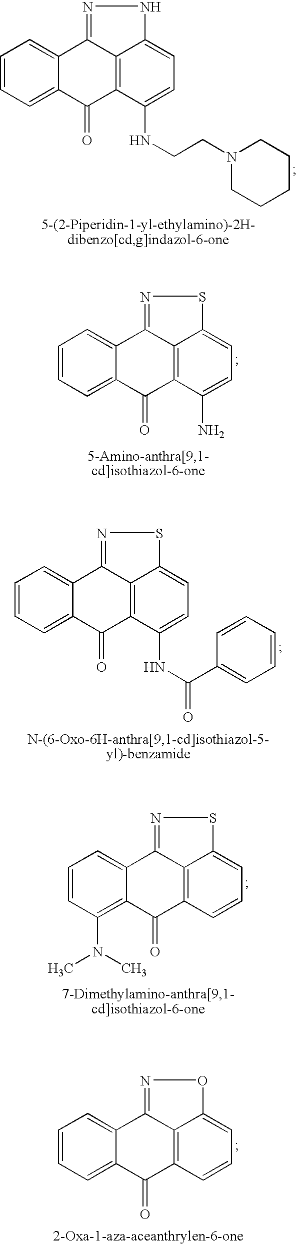
and pharmaceutically acceptable salts thereof.

Other JNK inhibitors that are useful in the present methods include, but are not limited to, those disclosed in International Publication No. WO 00/39101, (particularly at page 2, line 10 to page 6, line 12); International Publication No. WO 01/14375 (particularly at page 2, line 4 to page 4, line 4); International Publication No. WO 00/56738 (particularly at page 3, line 25 to page 6, line 13); International Publication No. WO 01/27089 (particularly at page 3, line 7 to page 5, line 29); International Publication No. WO 00/12468 (particularly at page 2, line 10 to page 4, line 14); European Patent Publication 1 110 957 (particularly at page 19, line 52 to page 21, line 9); International Publication No. WO 00/75118 (particularly at page 8, line 10 to page 11, line 26); International Publication No. WO 01/12621 (particularly at page 8, line 10 to page 10, line 7); International Publication No. WO 00/64872 (particularly at page 9, line 1 to page, 106, line 2); International Publication No. WO 01/23378 (particularly at page 90, line 1 to page 91, line 11); International Publication No. WO 02/16359 (particularly at page 163, line 1 to page 164, line 25); U.S. Pat. No. 6,288,089 (particularly at column 22, line 25 to column 25, line 35); U.S. Pat. No. 6,307,056 (particularly at column 63, line 29 to column 66, line 12); International Publication No. WO 00/35921 (particularly at page 23, line 5 to page 26, line 14); International Publication No. WO 01/91749 (particularly at page 29, lines 1-22); International Publication No. WO 01/56993 (particularly in at page 43 to page 45); and International Publication No. WO 01/58448 (particularly in at page 39), each of which is incorporated by reference herein in its entirety.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

4.3. Second Active Agents

As discussed above, a second active ingredient or agent can be used in the methods and compositions of the invention together with a JNK inhibitor to treat, prevent or manage CNS injury/damage and related syndromes. Specific second active agents can improve motor function and sensation in patients with CNS injury/damage and related syndromes, or prevent patient complications.

In one embodiment, the second active agent is steroids such as glucocorticoids, for example, but not limited to, methylprednisolone, dexamethasone and betamethasone.

In another embodiment, the second active agent is an anti-inflammatory agent, including, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHₒ-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone.

In another embodiment, the second active agent is a cAMP analog including, but not limited to, db-cAMP. Without being limited by theory, it is believed that certain JNK inhibitors and cAMP analogs can act in complementary or synergistic ways in the treatment or management of the disorders. It is also believed that the combined use of such agents may increase cAMP levels, enhance axonal sparing, myelination and growth of serotonergic fibers, and improve locomotion.

In another embodiment, the second active agent comprises a methylphenidate drug. In one embodiment, the methylphenidate drug comprises l-threo-methylphenidate, substantially free of any other piperidine. In one embodiment, the methylphenidate drug comprises d-threo-methylphenidate, substantially free of any other piperidine. In one embodiment, the methylphenidate drug comprises l-erythro-methylphenidate, substantially free of any other piperidine. In one embodiment, the methylphenidate drug comprises d-erythro-methylphenidate, substantially free of any other piperidine. In one embodiment, the methylphenidate drug comprises dl-threo-methylphenidate. In one embodiment, the methylphenidate drug comprises dl-erythro-methylphenidate. In one embodiment, the methylphenidate drug comprises some mixture of two or more of l-threo-methylphenidate, d-threo-methylphenidate, d-erythro-methylphenidate, and l-erytho-methylphenidate. In one embodiment, when a methylphenidate drug is used to treat CNS injury/damage and related syndromes, the administration of dosage forms, which contain an immediate dosage and a delayed second dosage, may provide for reduced abuse potential, improved convenience of administration, and better patient compliance. Certain dosage forms (e.g., pulsatile, pellets and bolus) and methods of administration of methylphenidate (e.g., d-threo-methylphenidate) are disclosed in U.S. Pat. Nos. 5,837,284 and 6,602,887, both of which are incorporated herein by reference in their entirety.

In another embodiment, the second active agent is diuretics. Diuretics are useful in decreasing brain volume and intracranial pressure (ICP). Mannitol, furosemide, glycerol and urea are commonly used. Metabolic therapies are also designed to decrease ICP by reducing the cerebral metabolic rate. Barbiturates are the most common class of drugs used to suppress cerebral metabolism.

In even another embodiment, the second active agent includes immunomodulatory agents, immunosuppressive agents, antihypertensives, anticonvulsants, fibrinolytic agents, antiplatelet agents, antipsychotics, antidepressants, benzodiazepines, buspirone, amantadine, and other known or conventional agents used in patients with CNS injury/damage and related syndromes.

In another embodiment, the second active agent is an IMiD® or a SelCID® (Celgene Corporation, New Jersey) (e.g., those disclosed in U.S. Pat. Nos. 6,075,041; 5,877,200; 5,698,579; 5,703,098; 6,429,221; 5,736,570; 5,658,940; 5,728,845; 5,728,844; 6,262,101; 6,020,358; 5,929,117; 6,326,388; 6,281,230; 5,635,517; 5,798,368; 6,395,754; 5,955,476; 6,403,613; 6,380,239; and 6,458,810, each of which is incorporated herein by reference in its entirety).

Surgical intervention such as decompressive craniectomy may be used in patients with refractory ICP elevation. In the surgical procedure, a large section of the skull is removed and the dura is expanded. This increases the total intracranial volume and, thus, decreases ICP.

In another embodiment, JNK inhibitors can be used in conjunction with neural transplantation to treat CNS injury/damage and related syndromes.

4.4. Methods of Treatments and Prevention

Methods of this invention encompass methods of preventing, treating and/or managing CNS injury/damage and related syndromes. CNS injury/damage and related syndromes include, but are not limited to, primary brain injury, secondary brain injury, traumatic brain injury, focal brain injury, diffuse axonal injury, head injury, concussion, post-concussion syndrome, cerebral contusion and laceration, subdural hematoma, epidermal hematoma, post-traumatic epilepsy, chronic vegetative state, complete SCI, incomplete SCI, acute SCI, subacute SCI, chronic SCI, central cord syndrome, Brown-Sequard syndrome, anterior cord syndrome, conus medullaris syndrome, cauda equina syndrome, neurogenic shock, spinal shock, altered level of consciousness, headache, nausea, emesis, memory loss, dizziness, diplopia, blurred vision, emotional lability, sleep disturbances, irritability, inability to concentrate, nervousness, behavioral impairment, cognitive deficit, and seizure.

As used herein, unless otherwise specified, the term “treating” refers to the administration of a composition after the onset of symptoms of CNS injury/damage and related syndromes, whereas “preventing” refers to the administration prior to the onset of symptoms, particularly to patients at risk of CNS injury/damage and related syndromes. As used herein, unless otherwise specified, the term “preventing” includes but is not limited to, inhibition or the averting of symptoms associated with CNS injury/damage and related syndromes. As used herein and unless otherwise indicated, the term “managing” encompasses preventing the recurrence of symptoms of CNS injury/damage and related syndromes in a patient who had suffered from CNS injury/damage and related syndromes, lengthening the time the symptoms remain in remission in a patient who had suffered from CNS injury/damage and related syndromes, and/or preventing the occurrence of CNS injury/damage and related syndromes in patients at risk of suffering from CNS injury/damage and related syndromes.

The symptoms associated with CNS injury/damage and related syndromes include, but are not limited to, motor weakness (especially paraparesis or quadriparesis with or without respiratory distress); loss of sensation or bowel or bladder control; sexual dysfunction; symptoms of neurogenic shock such as lightheadedness, diaphoresis, bradycardia, hypothermia, hypotension without compensatory tachycardia; pain; respiratory insufficiency; quadriplegia with upper and lower extremity areflexia; anesthesia below the affected level; loss of rectal and bladder sphincter tone; urinary and bowel retention leading to abdominal distention, ileus, and delayed gastric emptying; ipsilateral ptosis, miosis, anhydrosis; paralysis with loss of pain and temperature sensation; relative sparing of touch, vibration, and proprioception; dissociated sensory loss; arm weakness, patch sensory loss below the level of the lesion; loss of vibration and position sense below the level of the lesion, hyperreflexia, and an extensor toe sign; ipsilateral segmental anesthesia; and polyradiculopathy with pain, radicular sensory changes, asymmetric lower motor neuron-type leg weakness, and sphincter disturbances.

Methods encompassed by this invention comprise administering one or more JNK inhibitors, or a pharmaceutically acceptable salt, solvate, hydrate, stereoisomer, clathrate, or prodrug thereof to a patient (e.g., a human) suffering, or likely to suffer, from CNS injury/damage and related syndromes.

Another method comprises administering 1) a JNK inhibitor, or a pharmaceutically acceptable salt, solvate, hydrate, stereoisomer, clathrate, or prodrug thereof, and 2) a second active agent or active ingredient. Examples of JNK inhibitors are disclosed herein (see, e.g., section 4.2); and examples of the second active agents are also disclosed herein (see, e.g., section 4.3).

Administration of JNK inhibitors and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A specific route of administration for a JNK inhibitor is orally. Specific routes of administration for the second active agents or ingredients of the invention are known to those of ordinary skill in the art.

In one embodiment of the invention, the recommended daily dose range of a JNK inhibitor for the conditions described herein lie within the range of from about 1 mg to about 10,000 mg per day, given as a single once-a-day dose, or preferably in divided doses throughout a day. More specifically, the daily dose is administered twice daily in equally divided doses. Specifically, a daily dose range should be from about 1 mg to about 5,000 mg per day, more specifically, between about 10 mg and about 2,500 mg per day, between about 100 mg and about 800 mg per day, between about 100 mg and about 1,200 mg per day, or between about 25 mg and about 2,500 mg per day. In managing the patient, the therapy should be initiated at a lower dose, perhaps about 1 mg to about 2,500 mg, and increased if necessary up to about 200 mg to about 5,000 mg per day as either a single dose or divided doses, depending on the patient's global response.

4.4.1. Combination Therapy with a Second Active Agent

Specific methods of the invention comprise administering a JNK inhibitor, or a pharmaceutically acceptable salt, solvate, hydrate, stereoisomer, clathrate, or prodrug thereof, in combination with one or more second active agents, surgery or neural transplants. Examples of JNK inhibitors are disclosed herein (see, e.g., section 4.2). Examples of second active agents are also disclosed herein (see, e.g., section 4.3).

Administration of the JNK inhibitors and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A specific route of administration for a JNK inhibitor of the invention is oral. Specific routes of administration for the second active agents or ingredients of the invention are known to those of ordinary skill in the art. See, e.g., Physicians' Desk Reference, 1755-1760 (56^{th} ed., 2002).

In one embodiment of the invention, the second active agent is administered orally, intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount(s) of JNK inhibitors and any optional additional active agents concurrently administered to the patient. In a particular embodiment, the second active agent is methylprednisolone, dexamethasone, db-cAMP or a combination thereof.

In one embodiment, methylprednisolone can be administered in an amount of 30 mg/kg IV bolus over 15 minutes, followed by 5.4 mg/kg/h over 23 hours; and then IV infusion 45 minutes after conclusion of bolus.

In one embodiment, methylphenidate can be administered in an amount of from about 0.01 mg/kg to about 1 mg/kg.

In another embodiment, dexamethasone may be administered in an amount of from about 10-100 mg IV, followed by 6-10 mg IV every six hours for 24 hours.

In a specific embodiment of this method, a JNK inhibitor and db-cAMP can be administered to patients with CNS injury/damage and related syndromes.

4.4.2. Use with Transplantation Therapy

The invention encompasses a method of treating, preventing and/or managing CNS injury/damage and related syndromes, which comprises administering a JNK inhibitor, or a pharmaceutically acceptable salt, solvate, hydrate, stereoisomer, clathrate, or prodrug thereof, in conjunction with neural transplantation and stem cell transplantation.

Without being limited by theory, it is believed that the combined use of a JNK inhibitor and transplantation of Schwann cell or stem cell may provide additive or synergistic effects in patients with CNS injury/damage and related syndromes. In particular, it is believed that when used with transplanting Schwann cell or stem cell, a JNK inhibitor promotes significant supraspinal and proprioceptive axon sparing and myelination.

This invention encompasses a method of treating, preventing and/or managing CNS injury/damage and related syndromes which comprises administering to a patient (e.g., a human) a JNK inhibitor, or a pharmaceutically acceptable salt, solvate, hydrate, stereoisomer, clathrate, or prodrug thereof, before, during, or after surgery or the transplantation of Schwann cells or stem cells.

4.5. Pharmaceutical Compositions

Pharmaceutical compositions can be used in the preparation of individual, single unit dosage forms. Pharmaceutical compositions and dosage forms of the invention comprise a JNK inhibitor, or a pharmaceutically acceptable salt, solvate, hydrate, stereoisomer, clathrate, or prodrug thereof. Pharmaceutical compositions and dosage forms of the invention can further comprise one or more excipients.

Pharmaceutical compositions and dosage forms of the invention can also comprise one or more additional active agents. Consequently, pharmaceutical compositions and dosage forms of the invention comprise the active agents disclosed herein (e.g., a JNK inhibitor and a second active agent). Examples of optional second, or additional, active agents are disclosed herein (see, e.g., section 4.3).

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active agents it comprises than a dosage form used in the chronic treatment of the same disease. Similarly, a parenteral dosage form may contain smaller amounts of one or more of the active agents it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active agents in the dosage form. For example, the decomposition of some active agents may be accelerated by some excipients such as lactose, or when exposed to water. Active agents that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Specific lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise a JNK inhibitor, or a pharmaceutically acceptable salt, solvate, hydrate, stereoisomer, clathrate, or prodrug thereof in an amount of from about 1 to about 1,200 mg. Typical dosage forms comprise a JNK inhibitor, or a pharmaceutically acceptable salt, solvate, hydrate, stereoisomer, clathrate, or prodrug thereof in an amount of about 1, 2, 5, 10, 25, 50, 100, 200, 400, 800, 1,200, 2,500, 5,000 or 10,000 mg. In a particular embodiment, a specific dosage form comprises a JNK inhibitor in an amount of about 400, 800 or 1,200 mg. Typical dosage forms comprise the second active ingredient in an amount of 1 to about 1000 mg, from about 5 to about 500 mg, from about 10 to about 350 mg, or from about 50 to about 200 mg. Of course, the specific amount of the second active ingredient will depend on the specific agent used, the disorder being treated or managed, and the amount(s) of a JNK inhibitors and any optional additional active agents concurrently administered to the patient.

4.5.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A specific solid oral dosage form of the invention comprises a JNK inhibitor of the invention, anhydrous lactose, microcrystalline cellulose, polyvinylpyrrolidone, stearic acid, colloidal anhydrous silica, and gelatin.

4.5.2. Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.5.3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of a JNK inhibitor and its derivatives. See, e.g., U.S. Pat. No. 5,134,127, which is incorporated herein by reference.

4.5.4. Topical and Mucosal Dosage Forms

Topical and mucosal dosage forms of the invention include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.5.5. Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a dosage form of a JNK inhibitor, or a pharmaceutically acceptable salt, solvate, hydrate, stereoisomer, prodrug, or clathrate thereof. Kits encompassed by this invention can further comprise additional active agents. Examples of the additional active agents include, but are not limited to, those disclosed herein (see, e.g., section 4.3).

Kits of the invention can further comprise devices that are used to administer the active ingredients. Examples of such devices include, but are not limited to, syringes, drip bags, patches, and inhalers.

Kits of the invention can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5. EXAMPLES

Certain embodiments of the invention are illustrated by the following non-limiting examples.

5.1. Pharmacology Studies

A series of non-clinical pharmacology studies can be performed to support the clinical evaluation of a JNK inhibitor in human subjects. These studies are performed in accordance with internationally recognized guidelines for study design and in compliance with the requirements of Good Laboratory Practice (GLP), unless otherwise noted.

The pharmacological properties of a JNK inhibitor, including activity comparisons with thalidomide, are characterized in in vitro studies. Studies examine the effects of a JNK inhibitor on the production of various cytokines. In addition, a safety pharmacology study of a JNK inhibitor can be conducted in dogs and the effects of the compound on ECG parameters are examined further as part of three repeat-dose toxicity studies in primates.

5.2. Toxicology Studies

The effects of a JNK inhibitor on cardiovascular and respiratory function can be investigated in anesthetized dogs. Two groups of Beagle dogs (2/sex/group) are used. One group receives three doses of vehicle only and the other receives three ascending doses of a JNK inhibitor (400, 800, and 1,200 mg/kg/day). In all cases, doses of a JNK inhibitor or vehicle are successively administered via infusion through the jugular vein separated by intervals of at least 30 minutes.

5.3. Modulation of Cytokine Production

Inhibition of TNF-α production following LPS-stimulation of human PBMC and human whole blood by a JNK inhibitor can be investigated in vitro (Muller et al., Bioorg. Med. Chem. Lett. 9:1625-1630, 1999). The IC₅₀s of a JNK inhibitor for inhibiting production of TNF-α following LPS-stimulation of PBMC and human whole blood is measured.

For example, a JNK inhibitor can be tested for the ability to inhibit LPS-induced TNF-α production from human PBMC as previously described (Muller et al. 1996, J. Med. Chem. 39:3238). PBMC from normal donors is obtained by Ficoll Hypaque (Pharmacia, Piscataway, N.J., USA) density centrifugation. Cells are cultured in RPMI (Life Technologies, Grand Island, N.Y., USA) supplemented with 10% AB±human serum (Gemini Bio-products, Woodland, Calif., USA), 2 mM L-glutamine, 100 U/ml penicillin, and 100 μg/ml streptomycin (Life Technologies).

PBMC (2×10⁵ cells) are plated in 96-well flat-bottom Costar tissue culture plates (Corning, N.Y., USA) in triplicate. Cells are stimulated with LPS (Sigma, St. Louis, Mo., USA) at 100 ng/ml in the absence or presence of compounds. a JNK inhibitor is dissolved in DMSO (Sigma) and further dilutions are done in culture medium immediately before use. The final DMSO concentration in the sample is 0.25%. The a JNK inhibitor is added to cells 1 hour before LPS stimulation. Cells are incubated for 18-20 hours at 37° C. in 5% CO₂ and supernatants are then collected, diluted with culture medium and assayed for TNF-α levels by ELISA (Endogen, Boston, Mass., USA).

During the course of inflammatory diseases, TNF-α production is often stimulated by the cytokine 1L-1β, rather than by bacterially derived LPS. A JNK inhibitor can be tested for the ability to inhibit IL-1β-induced TNF-α production from human PBMC as described above for LPS-induced TNF-α production, except that the PBMC is isolated from source leukocyte units (Sera-Tec Biologicals, North Brunswick, N.J., USA) by centrifugation on Ficoll-Paque Plus (Amersham Pharmacia, Piscataway, N.J., USA), plated in 96-well tissue culture plates at 3×10⁵ cells/well in RPMI-1640 medium (BioWhittaker, Walkersville, Md., USA) containing 10% heat-inactivated fetal bovine serum (Hyclone), 2 mM L-glutamine, 100 U/ml penicillin, and 100 mg/ml streptomycin (complete medium), pretreated with JNK inhibitors at 10, 2, 0.4, 0.08, 0.016, 0.0032, 0.00064, and 0 μM in duplicate at a final DMSO concentration of 0.1% at 37° C. in a humidified incubator at 5% CO₂ for 1 hour, then stimulated with 50 ng/ml recombinant human IL-1β (Endogen) for 18 hours.

5.4. Inhibition of JNK

The ability of a JNK inhibitor to inhibit JNK and accordingly, to be useful for the treatment, prevention, management and/or modification of a central nervous system injury/damage or related syndromes, can be demonstrated using one or more of the following assays. The assays were performed using the following illustrative JNK inhibitor: 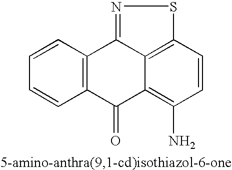
JNK Assay

To 10 μL of 5-amino-anthra(9,1-cd)isothiazol-6-one in 20% DMSO/80% dilution buffer containing of 20 mM HEPES (pH 7.6), 0.1 mM EDTA, 2.5 mM magnesium chloride, 0.004% Triton x100, 2 μg/mL leupeptin, 20 mM β-glycerolphosphate, 0.1 mM sodium vanadate, and 2 mM DTT in water was added 30 μL of 50-200 ng His6-JNK1, JNK2, or JNK3 in the same dilution buffer. The mixture was pre-incubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton x100, 11 μM ATP, and 0.5 μCi γ-32P ATP in water was added and the reaction was allowed to proceed for 1 hour at room temperature. The c-Jun phosphorylation was terminated by addition of 150 μL of 12.5% trichloroacetic acid. After 30 minutes, the precipitate was harvested onto a filter plate, diluted with 50 μL of the scintillation fluid and quantified by a counter. The IC₅₀ values were calculated as the concentration of 5-amino-anthra(9,1-cd)isothiazol-6-one at which the c-Jun phosphorylation was reduced to 50% of the control value. Compounds that inhibit JNK preferably have an IC₅₀ value ranging 0.01-10 μM in this assay. 5-Amino-anthra(9,1-cd)isothiazol-6-one has an IC₅₀ according to this assay of 1 μM for JNK2 and 400 nM for JNK3. The measured IC₅₀ value for 5-amino-anthra(9,1-cd)isothiazol-6-one, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one inhibits JNK. This assay demonstrates that 5-amino-anthra(9,1-cd)isothiazol-6-one, an illustrative JNK inhibitor, inhibits JNK2 and JNK3 and, accordingly, is useful for the treatment, prevention, management and/or modification of a central nervous system injury/damage or related syndromes.

Selectivity For JNK:

5-Amino-anthra(9,1-cd)isothiazol-6-one was also assayed for its inhibitory activity against several protein kinases, listed below, using techniques known to those skilled in art (See, e.g., Protein Phosphorylation, Sefton & Hunter, Eds., Academic Press, pp. 97-367, 1998). The following IC₅₀ values were obtained:

| Enzyme | IC₅₀ |
|---|---|
| p38-2 | >30,000 nM |
| MEK6 | >30,000 nM |
| LKK1 | >30,000 nM |
| IKK2 | >30,000 nM |

This assay shows that 5-amino-anthra(9,1-cd)isothiazol-6-one, an illustrative JNK inhibitor, selectively inhibits JNK relative to other protein kinases and, accordingly, is a selective JNK inhibitor. Therefore, 5-amino-anthra(9,1-cd)isothiazol-6-one, an illustrative JNK inhibitor, is useful for the treatment, prevention, management and/or modification of a central nervous system injury/damage or related syndromes.

Jurkat T-cell IL-2 Production Assay:

Jurkat T cells (clone E6-1) were purchased from the American Type Culture Collection of Manassas, Va. and maintained in growth media consisting of RPMI 1640 medium containing 2 mM L-glutamine (commercially available from Mediatech Inc. of Herndon, Va.), with 10% fetal bovine serum (commercially available from Hyclone Laboratories Inc. of Omaha, Nebr.) and penicillin/streptomycin. All cells were cultured at 37° C. in 95% air and 5% CO₂. Cells were plated at a density of 0.2×10⁶ cells per well in 200 μL of media. Compound stock (20 mM) was diluted in growth media and added to each well as a 10× concentrated solution in a volume of 25 μL, mixed, and allowed to pre-incubate with cells for 30 minutes. The compound vehicle (dimethylsulfoxide) was maintained at a final concentration of 0.5% in all samples. After 30 minutes the cells were activated with PMA (phorbol myristate acetate, final concentration 50 ng/mL) and PHA (phytohemagglutinin, final concentration 2 μg/mL). PMA and PHA were added as a 10× concentrated solution made up in growth media and added in a volume of 25 μL per well. Cell plates were cultured for 10 hours. Cells were pelleted by centrifugation and the media removed and stored at −20° C. Media aliquots are analyzed by sandwich ELISA for the presence of IL-2 as per the manufacturers instructions (Endogen Inc. of Woburn, Mass.). The IC₅₀ values were calculated as the concentration of 5-amino-anthra(9,1-cd)isothiazol-6-one at which the IL-2 production was reduced to 50% of the control value. Compounds that inhibit JNK preferably have an IC₅₀ value ranging from 0.1-30 μM in this assay. 5-Amino-anthra(9,1-cd)isothiazol-6-one has an IC₅₀ of 30 μM. The measured IC₅₀ value for 5-amino-anthra(9,1-cd)isothiazol-6-one, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one inhibits JNK.

This assay shows that 5-amino-anthra(9,1-cd)isothiazol-6-one, an illustrative JNK inhibitor, inhibits IL-2 production in Jurkat T-cells and accordingly inhibits JNK. Therefore, 5-amino-anthra(9,1-cd)isothiazol-6-one, an illustrative JNK inhibitor, is useful for the treatment, prevention, management and/or modification of a central nervous system injury/damage or related syndromes.

[³H]Dopamine Cell Culture Assay:

Cultures of dopaminergic neurons were prepared according to a modification of the procedure described by Raymon and Leslie (J. Neurochem. 62:1015-1024, 1994). Time-mated pregnant rats were sacrificed on embyronic day 14-15 (crown rump length 11-12 mm) and the embryos removed by cesarean section. The ventral mesencephalon, containing the dopaminergic neurons, was dissected from each embryo. Tissue pieces from approximately 48 embryos were pooled and dissociated both enzymatically and mechanically. An aliquot from the resulting cell suspension was counted and the cells were plated in high glucose DMEM/F12 culture medium with 10% fetal bovine serum at a density of 1×10⁵ cells/well of a Biocoat poly-D-lysine-coated 96-well plate. The day following plating was considered 1 day in vitro (DIV). Cells were maintained in a stable environment at 37° C., 95% humidity, and 5% CO₂. A partial medium change was performed at 3 DIV. At 7 DIV, cells were treated with the neurotoxin, 6-hydroxydopamine (6-OHDA, 30 μM) in the presence and absence of 5-amino-anthra(9,1-cd)isothiazol-6-one. Cultures were processed for [³H]dopamine uptake 22 hours later.

[³H]Dopamine uptake is used as a measure of the health and integrity of dopaminergic neurons in culture (Prochiantz et al., PNAS 76: 5387-5391, 1979). It was used in these studies to monitor the viability of dopaminergic neurons following exposure to the neurotoxin 6-OHDA. 6-OHDA has been shown to damage dopaminergic neurons both in vitro and in vivo and is used to model the cell death observed in Parkinson's disease (Ungerstedt, U., Eur. J. Pharm., 5 (1968) 107-110 and Hefti et al., Brain Res., 195 (1980) 123-137). Briefly, cells treated with 6-OHDA in the presence and absence of 5-amino-anthra(9,1-cd)isothiazol-6-one were assessed in the uptake assay 22 hrs after exposure to 6-OHDA. Culture medium was removed and replaced with warm phosphate buffered saline (PBS) with calcium and magnesium, 10 μM pargyline, 1 mM ascorbic acid, and 50 nM [³H]dopamine. Cultures were incubated at 37° C. for 20 min. Radioactivity was removed and the cultures were washed 3× with ice cold PBS. To determine the intracellular accumulation of [³H]dopamine, cells were lysed with M-PER detergent and an aliquot was taken for liquid scintillation counting. The measured effect of 5-amino-anthra(9,1-cd) isothiazol-6-one on the intracellular accumulation of [³H]dopamine, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one protects rat ventral mesencephalan neurons from the toxic effects of 6-OHDA. Accordingly, 5-amino-anthra(9,1-cd)isothiazol-6-one, an illustrative JNK inhibitor, is useful for the treatment, prevention, management and/or modification of a central nervous system injury/damage or related syndromes.

Brain-Blood Plasma Distribution of 5-amino-anthra(9,1-cd)isothiazol-6-one In Vivo

5-Amino-anthra(9,1-cd)isothiazol-6-one was administered intravenously (10 mg/kg) into the veins of Sprague-Dawley rats. After 2 hr, blood samples were obtained from the animals and their vascular systems were perfused with approximately 100 mL of saline to rid their brains of blood. The brains were removed from the animals, weighed, and homogenized in a 50 mL conical tube containing 10 equivalents (w/v) of methanol/saline (1:1) using a Tissue Tearer (Fischer Scientific). The homogenized material was extracted by adding 600 μL of cold methanol to 250 μL of brain homogenate vortexed for 30 sec and subjected to centrifugation for 5 min. After centrifugation, 600 μL of the resulting supernatant was transferred to a clean tube and evaporated at room temperature under reduced pressure to provide a pellet. The resulting pellet was reconstituted in 250 μL of 30% aqueous methanol to provide a brain homogenate analysis sample. A plasma analysis sample was obtained using the brain homogenate analysis sample procedure described above by substituting plasma for brain homogenate. Standard plasma samples and standard brain homogenate samples containing known amounts of 5-amino-anthra(9,1-cd)isothiazol-6-one were also prepared by adding 5 μL of serial dilutions (50:1) of a solution of 5-amino-anthra(9,1-cd)isothiazol-6-one freshly prepared in cold ethanol to 250 μL of control rat plasma (Bioreclamation of Hicksville, N.Y.) or control brain homogenate. The standard plasma samples and standard brain homogenate samples were then subjected to the same extraction by protein precipitation, centrifugation, evaporation, and reconstitution procedure used for the brain homogenate to provide brain homogenate standard analysis samples and plasma standard analysis samples. The brain homogenate analysis samples, plasma analysis samples, and standard analysis samples were analyzed and compared using HPLC by injecting 100 μL of a sample onto a 5 μm C-18 Luna column (4.6 mm×150 mm, commercially available from Phenomenex of Torrance, Calif.) and eluting at 1 mL/min with a linear gradient of 30% aqueous acetonitrile containing 0.1% trifluoroacetic acid to 90% aqueous acetonitrile containing 0.1% trifluoroacetic acid over 8 minutes and holding at 90% aqueous acetonitrile containing 0.1% trifluoroacetic acid for 3 min. with absorbance detection at 450 nm. Recovery of 5-amino-anthra(9,1-cd)isothiazol-6-one was 56±5.7% for plasma and 42±6.2% for the brain. The concentration of 5-amino-anthra(9,1-cd) isothiazol-6-one in the brain and plasma was determined by comparing HPLC chromatograms obtained from the brain homogenate analysis samples and plasma analysis samples to standard curves constructed from analysis of the brain homogenate standard analysis samples and the plasma standard analysis samples, respectively. Results from this study show that 5-amino-anthra(9,1-cd)isothiazol-6-one, following intravenous administration, crosses the blood-brain barrier to a significant extent. In particular, brain-drug concentrations were approximately 65 nmole/g and plasma concentrations were approximately 7 μM at 2 hr post-dose, resulting in a brain-plasma concentration ratio of approximately 9-fold (assuming 1 g of brain tissue is equivalent to 1 mL of plasma). This example shows that 5-amino-anthra(9,1-cd)isothiazol-6-one, an illustrative JNK inhibitor, has enhanced ability to cross the blood-brain barrier. In addition, this example shows that the JNK inhibitors, in particular 5-amino-anthra(9,1-cd)isothiazol-6-one, can cross the blood-brain barrier when administered to a patient.

5.5. Clinical Studies

Patients with CNS injury/damage are treated with a JNK inhibitor (about 1 to 5,000 mg orally daily). For example, a JNK inhibitor is administered alone or in combination with prednisolone or dexamethasone.

The embodiments of the invention described herein are intended to be merely exemplary, and those skilled in the art will recognize, or will be able to ascertain using no more than routine experimentation, numerous equivalents of specific compounds, materials, and procedures. All such equivalents are considered to be within the scope of the invention.

What is claimed is:

1. A method of treating or preventing a central nervous system injury, which comprises administering to a patient in need of such treatment or prevention a therapeutically or prophylactically effective amount of a JNK inhibitor or a pharmaceutically acceptable salt, solvate, or stereoisomer thereof.

2. A method of claim 1, wherein the central nervous system injury is primary brain injury, secondary brain injury, traumatic brain injury, focal brain injury, diffuse axonal injury, head injury, concussion, post-concussion syndrome, cerebral contusion and laceration, subdural hematoma, epidermal hematoma, post-traumatic epilepsy, chronic vegetative state, complete spinal cord injury, incomplete spinal cord injury, acute spinal cord injury, subacute spinal cord injury, chronic spinal cord injury, central cord syndrome, Brown-Sequard syndrome, anterior cord syndrome, conus medullaris syndrome, cauda equina syndrome, neurogenic shock or spinal shock.

3. A method of claim 1, further comprising administering a therapeutically or prophylactically effective amount of a second active agent.

4. A method of managing a central nervous system injury, which comprises administering to a patient in need of such management a prophylactically effective amount of a JNK inhibitor, or a pharmaceutically acceptable salt, solvate, or stereoisomer thereof.

5. A method of claim 4, wherein the central nervous system injury is primary brain injury, secondary brain injury, traumatic brain injury, focal brain injury, diffuse axonal injury, head injury, concussion, post-concussion syndrome, cerebral contusion and laceration, subdural hematoma, epidermal hematoma, post-traumatic epilepsy, chronic vegetative state, complete spinal cord injury, incomplete spinal cord injury, acute spinal cord injury, subacute spinal cord injury, chronic spinal cord injury, central cord syndrome, Brown-Sequard syndrome, anterior cord syndrome, conus medullaris syndrome, cauda equina syndrome, neurogenic shock or spinal shock.

6. A method of claim 4, further comprising administering a therapeutically or prophylactically effective amount of a second active agent

7. A method of claim 3 or 6, wherein the second active agent is an anti-inflammatory agent, a steroid, a cAMP analog, an antihypertensive agent, an anticonvulsant agent, a fibrinolytic agent, an antiplatelet agent, an antipsychotic agent, an antidepressant agent, a benzodiazepine, a buspirone, a stimulant, an amantadine, a diuretic, a barbiturate, an immunosuppressive agent or an immunomodulatory agent.

8. A method of claim 1 or 4, wherein the stereoisomer of the JNK inhibitor is enantiomerically pure.

9. A method of treating or preventing a central nervous system injury, which comprises administering to a patient in need of such treatment or prevention a therapeutically or prophylactically effective amount of a compound having the formula: 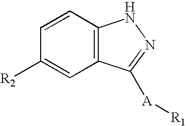
or a pharmaceutically acceptable salt, solvate or stereoisomer thereof,
wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)CC(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, substituted aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

10. A method of claim 9, wherein the compound is: 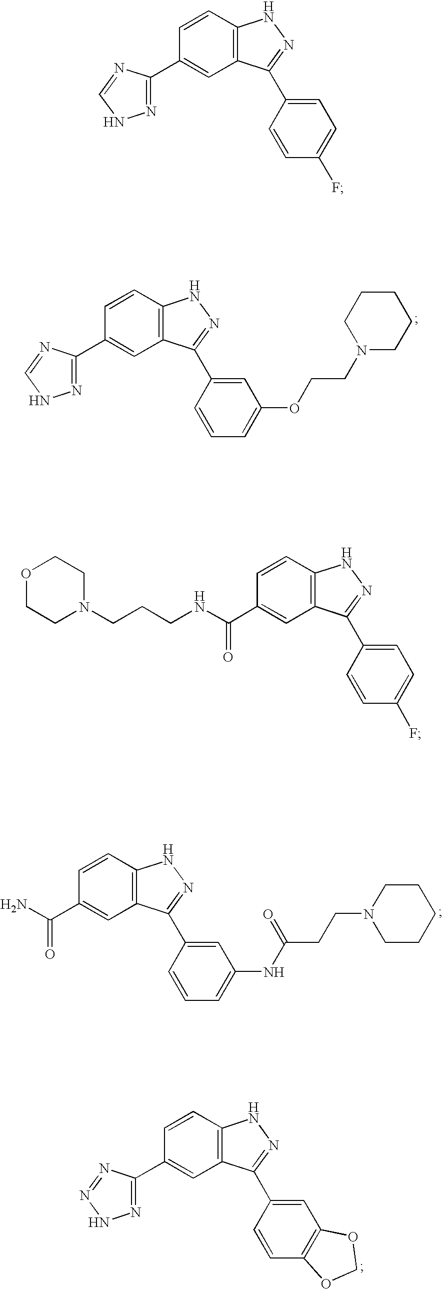 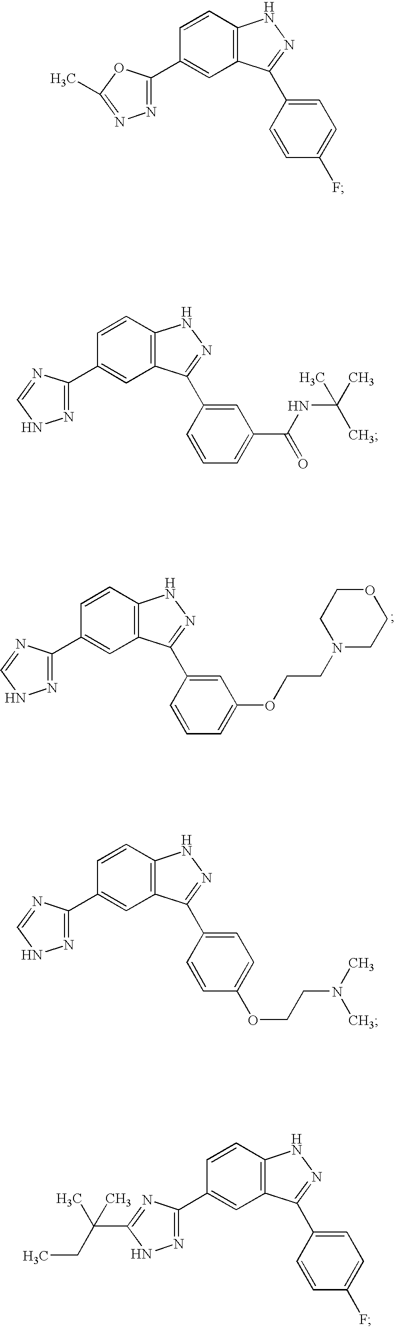 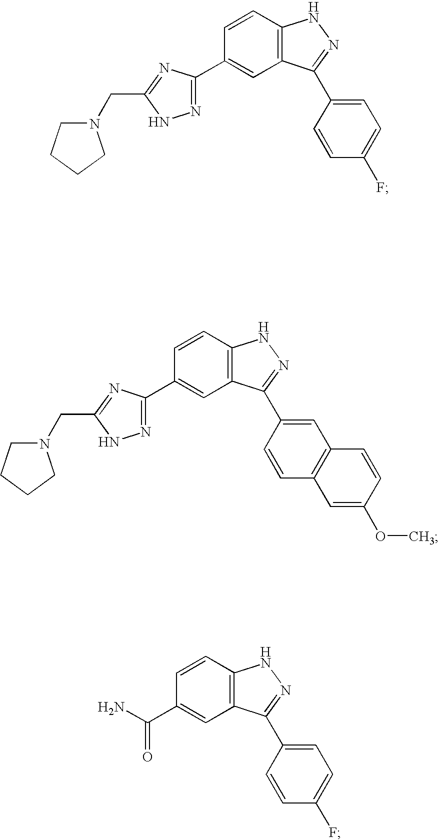
or a pharmaceutically acceptable salt, solvate or stereoisomer thereof.

11. A method of treating or preventing a central nervous system injury, which comprises administering to a patient in need of such treatment or prevention a therapeutically or prophylactically effective amount of a compound having the formula: 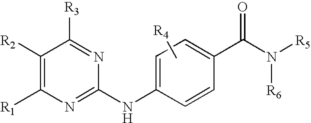
or a pharmaceutically acceptable salt, solvate or stereoisomer thereof,
wherein:
R₁ is aryl or heteroaryl optionally substituted with one to four substituents independently selected from R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, —(CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, substituted alkyl, aryl, arylalkyl, heterocycle or heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

12. A method of claim 11, wherein the compound is: 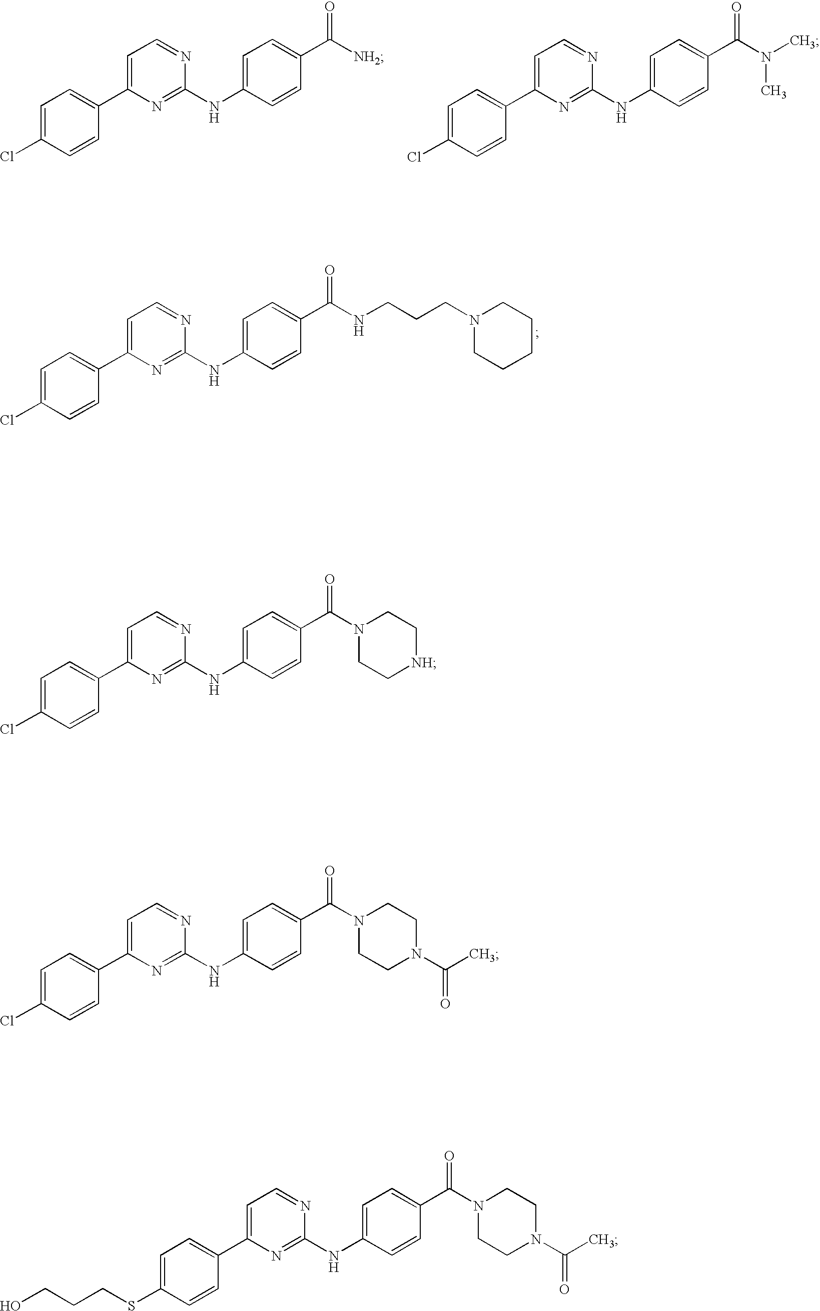 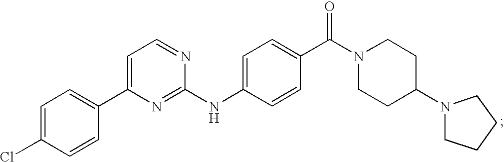
or a pharmaceutically acceptable salt, solvate or stereoisomer thereof.

13. A method of treating or preventing a central nervous system injury, which comprises administering to a patient in need of such treatment or prevention a therapeutically or prophylactically effective amount of a compound having the formula: 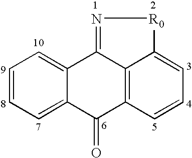
or a pharmaceutically acceptable salt, solvate or stereoisomer thereof,
wherein
R₀ is —O—, —S—, —S(O)—, —S(O)₂—, NH or —CH₂—;
the compound being (i) unsubstituted, (ii) monosubstituted and having a first substituent, or (iii) disubstituted and having a first substituent and a second substituent; 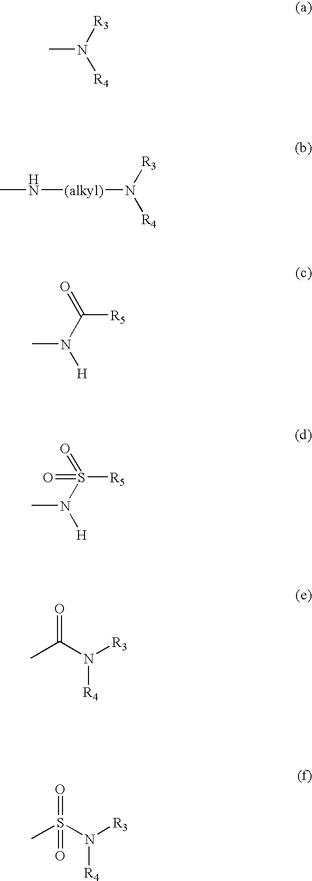
the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by formula (a), (b), (c), (d), (e), or (f):
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

14. A method of claim 13, wherein the compound is: 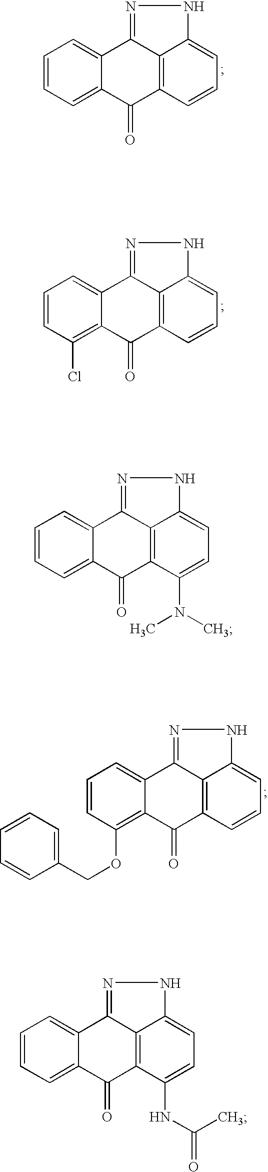 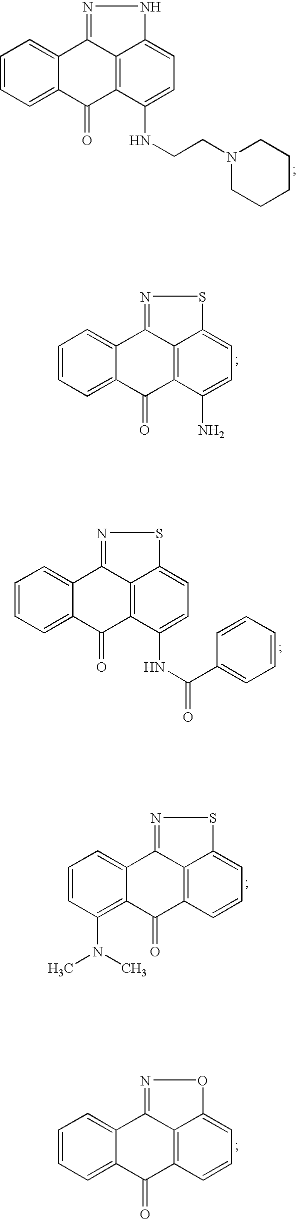
or a pharmaceutically acceptable salt, solvate or stereoisomer thereof.

15. A method of reducing or avoiding an adverse effect associated with the administration of a second active agent in a patient suffering from a central nervous system injury, which comprises administering to a patient in need of such reduction or avoidance an effective amount of the second active agent and a therapeutically or prophylactically effective amount of a JNK inhibitor, or a pharmaceutically acceptable salt, solvate, or stereoisomer thereof.

16. A method of claim 15, wherein the second active agent is an anti-inflammatory agent, a steroid, a cAMP analog, an antihypertensive agent, an anticonvulsant agent, a fibrinolytic agent, an antiplatelet agent, an antipsychotic agent, an antidepressant agent, a benzodiazepine, a buspirone, a stimulant, an amantadine, a diuretic, a barbiturate, an immunosuppressive agent or an immunomodulatory agent.

17. A pharmaceutical composition comprising a JNK inhibitor, or a pharmaceutically acceptable salt, solvate, or stereoisomer thereof, in an amount effective to treat, prevent or manage a central nervous system injury, and an effective amount of a second active agent useful for treating or preventing a central nervous system injury.

18. A pharmaceutical composition of claim 17, wherein the second active agent is an anti-inflammatory agent, a steroid, a cAMP analog, an antihypertensive agent, an anticonvulsant agent, a fibrinolytic agent, an antiplatelet agent, an antipsychotic agent, an antidepressant agent, a benzodiazepine, a buspirone, a stimulant, an amantadine, a diuretic, a barbiturate, an immunosuppressive agent or an immunomodulatory agent.
